(19) United States
(12) Patent Application Publication
(10) Pub. No.: US 2024/0148859 A1
(43) Pub. Date: May 9, 2024

(54) MICROORGANISM DISPLAYING ANTIGENIC PROTEIN OF THE SARS-CoV2 CORONAVIRUS

(71) Applicant: LES BIOTECHNOLOGIES ULYSSE INC., Trois-Rivieres (CA)

(72) Inventors: Yves HURTUBISE, Trois-Rivieres (CA); Genevieve LAPERRIERE, Becancour (CA)

(21) Appl. No.: 18/281,416

(22) PCT Filed: Mar. 10, 2022
(86) PCT No.: PCT/CA2022/050351

§ 371 Date: Sep. 11, 2023

Related U.S. Application Data
(60) Provisional application No. 63/159,525, filed on Mar. 11, 2021.

(51) Int. Cl. A61K 39/215; A61P 31/14; C07K 14/005; C12N 1/20; C12N 7/00; C12N 15/75; A61K 39/00; C12R 1/125

(52) U.S. Cl. CPC A61K 39/215; A61P 31/14; C07K 14/005; C12N 1/205; C12N 7/00; C12N 15/75; A61K 2039/523; A61K 2039/575; C12N 2770/20034; C12R 2001/125

(57) ABSTRACT

The present technology generally relates to a microorganism displaying antigenic proteins of the SARS-CoV2 coronavirus on its surface, to methods of preparing same, to composition comprising such microorganism and to methods for treatment of SARS-CoV2 related infections in subjects.

### CROSS-REFERENCE TO RELATED APPLICATION

This application claims the benefit of and priority to U.S. provisional patent application No. 63/159,525, filed on Mar. 11, 2021; the content of which is herein incorporated in entirety by reference.

### FIELD OF TECHNOLOGY

The present technology generally relates to a microorganism displaying antigenic proteins of the SARS-CoV2 coronavirus on its surface, to methods of preparing same, to composition comprising such microorganism and to methods for treatment of SARS-CoV2 related infections in subjects.

### BACKGROUND INFORMATION

Vaccines may take different forms, but they are typically intended to allow an organism to develop an immune response without being exposed to a virulent pathogen agent. Approaches to vaccines include attenuated vaccines that use weakened virus that cannot reproduce in its host; inactive vaccines that use viruses that have been destroyed by heat or by chemical means; viral vector vaccines which use a non-dangerous virus which has had the necessary genetic material from the virus to be prevented injected into its basic genetic matter, in order to obtain an effective immune response; subunit vaccines that principally use peptides or proteins to be injected into the patient whose immune system is to be induced; vaccines that are based on nucleic acids DNA and RNA messenger that, in both cases, provide the genetic information necessary to the cells of the vaccinated patient in order to be translated into antigenic proteins to be recognized by the immune system. This latest technology had never been approved for use in humans before the pandemic associated with the SARS-CoV-2 virus. Since early 2021, the vaccines developed by Pfizer, Moderna (RNA messenger vaccine) and Astra Zeneca (DNA vaccine) have been the principal tools of prevention used to counter infection by the SARS-CoV-2 virus.

The massive investment by several national governments has facilitated the accelerated development of vaccines based on nucleic acids. In less than 18 months, Pfizer, Moderna, and Astra Zeneca began the mass production of a first genustion of a vaccine against SARS-CoV-2. Unfortunately, after 12 months of an intensive use of vaccines based on nucleic acids, 4 consecutive waves of SARS-CoV-2 followed. This required at least 3 vaccination campaigns in order to seek to maintain sufficient protection against the new variants.

Although it is very interesting, the technology of vaccines based on nucleic acids has certain disadvantages. The main disadvantages are the following: the need to maintain the vaccines between a temperature of 4° C. and −40° C.; the need to organize vaccination campaigns that require large adapted spaces and a large number of specialized persons; a high cost per dose making in inaccessible to impoverished countries; the impossibility of reaching more than 2.5 billion persons in impoverished countries; great difficulties in reaching remote populations in a large number of impoverished countries.

Another disadvantage of the technology based on nucleic acids relates to quality; its very important genetic code specificity used to produce a part of the antigen. In comparison to standard methods for creating vaccines, technology based on DNA and RNA messenger ultimately only provide a small antigen, while other methods provide one or more full antigens. This very targeted intervention is effective in the protection from serious symptoms of the illness but have a tendency to favor the appearance of a higher number of mutations specifically targeting this area, rendering the vaccine less and less effective [1].

Continual genetical analysis carried out since the beginning of the SARS-CoV-2 pandemic has made it possible to detect more than 7 variants (Alpha B1.1.7; Beta B.1.351; Gamma P1; Delta B1.617.2; Delta Plus; Omicron B.1.1.529 and Omicron B.2) as well as several other variants of interest. The current vaccines are based on a nucleotide sequence that codes for a part (Pfizer and Moderna with the RBD=receptor binding domain) or all of SARS-CoV-2 S Protein (Astra Zeneca). Knowing that most of the major mutations that have been detected in the different variants are in the S Protein, and more specifically in the receptor binding domain (RBD). Despite 3 doses of the vaccine, a significative percentage of vaccinated people can still develop a serious form of the illness.

In the context of a world pandemic, the World Health Organization has pleaded over the last several months in favor of the development of a new generation of vaccines that will make it possible to offer better protection of the population. In order to minimize the problematic of the increase of variants, it has become urgent to develop a more effective, less costly vaccine, that does not require the organization of complex and expensive vaccine campaigns, and that can be stored between 4 and 50° C. without loss of effectiveness.

The adaptation of protein cell display technology as a method of production for a new generation of vaccines is a very promising alternative that can respond to different expectations described by the WHO. This approach was created in 1990 and has been adapted since then for different food-grade microorganisms. This type of approach makes it possible to display different new functions on the surface of the microorganism of interest that are naturally absent in the host organism. This technology is used, among other uses, to display enzymes that will be used as natural catalyzers for producing biomolecules of interest. Enzymes (e.g., chitinases) that may be used to attack the presence of pathogenic fungus affecting the growth of plants can also be displayed [2]. The control of bacterial pathogens can also be carried out with success by displaying a specific bacteriocin [3]. This technology can be used in a multitude of applications including the production of antigens. In this case, the choice of the microorganism is very important, because it must be without danger for humans or animals. A good number of examples have been described in literature and it is upon this approach that the present disclosure is based.

### SUMMARY

It is an object of the present technology to ameliorate at least some of the deficiencies present in the prior art.

In some aspects, the present technology relates to a recombinant microorganism comprising an antigen of SARS-CoV-2 expressed on the surface of the microorganism, wherein the recombinant microorganism is capable of inducing an immune response against a SARS-CoV-2 infection in a subject.

In some aspects, the present technology relates to a recombinant microorganism displaying a fusion protein at its surface, wherein the fusion protein comprises: i) a receptor binding portion of a SARS-CoV-2 protein, ii) a multi-epitope chimeric portion of SARS-CoV-2 proteins; and a membranal protein of the microorganism; and wherein the recombinant microorganism is capable of inducing an immune response against a epitopes related to different SARS-CoV-2 proteins infection in a subject.

In some aspects, the present technology relates to a method for displaying an antigen of SARSCoV-2 on a surface of a microorganism, the method comprising the steps of: (i) preparing a vector for microorganism surface display comprising a gene construct, the gene construct comprising: i) a microorganism membrane attachment protein; ii) a gene encoding for an enzymatic activity that is absent in microorganism in the wild, and iii) a gene encoding the antigen of SARS-CoV-2, wherein, when expressed, the gene construct expresses a fusion protein between the a microorganism membrane attachment protein, the gene encoding for an enzymatic activity and the antigen of SARS-CoV-2; (ii) transforming a microorganism host cell with the vector for microorganism surface display; and (iii) displaying the antigen of SARS-CoV-2 on a surface of the host cell. IN some instances, the gene construct further comprises nucleic acid sequence encoding for a multi-epitope chimeric portion of SARS-CoV-2 proteins.

In some instances, the microorganism is a member of a Bacillus genus. In some instances, the microorganism is a spore from a member of a Bacillus genus such as for example: Bacillus subtilis, Bacillus circulans, Bacillus clausii, Bacillus amyloliquefaciens and Bacillus velezensis. 1N some instances, the Bacillus is Bacillus subtilis.

An isolated nucleic acid molecule encoding for a fusion protein, wherein the fusion protein comprises: i) a receptor binding portion of a SARS-CoV-2 protein, ii) a multi-epitope chimeric portion of SARS-CoV-2 proteins; and iii) a membranal protein of the microorganism.

In some aspects, the present technology relates to a composition comprising the recombinant microorganism according to any one of claims 1 to 19 and a pharmaceutically acceptable diluent, carrier or excipient.

In some aspects, the present technology relates to a vaccine for prevention or treatment of a Coronavirus infection in a subject, the vaccine comprising an effective amount of the recombinant microorganism or the composition as described herein.

In some aspects, the present technology relates to a method for treating a SARS-CoV-2 infection in a subject, the method comprising administering to the subject the recombinant microorganism as described herein; the composition as described herein; or the vaccine as described herein; such that Coronavirus infection is prevented or treated in the subject.

In some aspects, the present technology relates to a method of inducing immunity against a SARS-CoV-2 infection comprising administering to a subject the recombinant microorganism as described herein; the composition as described herein; or the vaccine as described herein; such that the SARS-CoV-2 infection is treated in the subject.

Other aspects and features of the present technology will become apparent to those ordinarily skilled in the art upon review of the following description of specific embodiments in conjunction with the accompanying drawings.

### BRIEF DESCRIPTION OF THE DRAWINGS

Further aspects and advantages of the present technology will become better understood with reference to the description in association with the following in which:

FIG. 1 is a schematic representation of the Bacillus sporulation process.

FIG. 2 is a schematic representation of a Bacillus spore vaccine display concept.

FIG. 3 is a schematic representation of principal Bacillus spore coat.

FIG. 4 is a schematic representation of a plasmid: pG-cscA (sucrose hydrolase) for selection with sucrose on minimal media for E. coli DH5α.

FIG. 5 is a picture of petri dishes showing selection of transformant of DH5α/pG-cscA on minimal media+sucrose. Control=DH5α.

FIG. 6 is a schematic representation of a plasmid: pG-Bvlnu (inulinase) for selection with inulin on minimal media for E. coli DH5α.

FIG. 7 is a picture of petri dishes showing a selection of transformant of DH5α/pG-Bvlnu on minimal media+inuline. Control=DH5α.

FIG. 8 is a schematic representation of a plasmid: pUB206-M-VF.

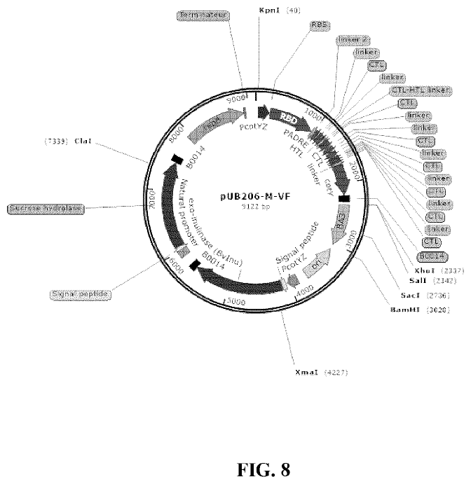

FIG. 9 is a schematic representation ofa plasmid: pUB205-P-VF.

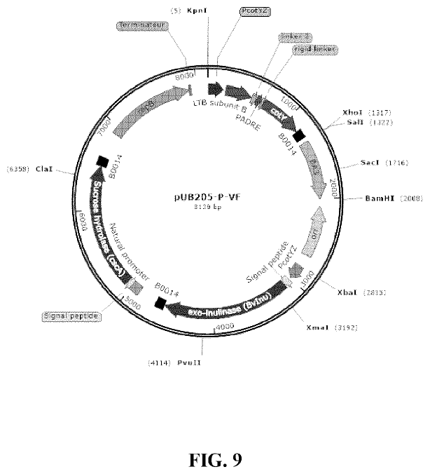

FIG. 10 is a picture of petri dishes showing a phospholipase activity detected around B. velezensis U50 transformed with pUB104.

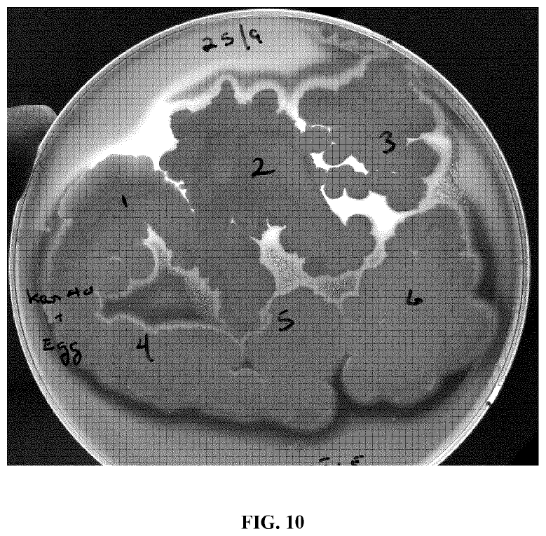

FIG. 11 is a picture of a petri dish showing the creation of “eternal spores” in B. subtilis U50.

FIG. 12 is a schematic representation of a plasmid: pUBSE2.

FIG. 13 is a schematic representation of a plasmid: pUB104.

FIG. 14 is a schematic representation of a plasmid: pUB103.

FIG. 15 is a schematic representation of a plasmid: pUB206_M.

FIG. 16 is a schematic representation of a plasmid: pUB205-P.

FIG. 17 is a schematic representation of a plasmid: pUB204_OM.

FIGS. 18A and 18B are graphic diagrams showing the results of analysis using flow cytometer for the expression of green fluorescent protein (GFP) on the surface of Bacillus 50 spores. Surface expression of the adjuvant E. coli Heat-labile enterotoxin B chain on the surface of Bacillus U148 vegetative cells. In red, the spores were stained with the secondary antibody only and in blue, with a mouse monoclonal antibody specific to GFP.

FIG. 19 is a graphic diagram showing the results of analysis using flow cytometer for the expression of Heat-labile enterotoxin B chain (LTB) on the surface of Bacillus U148 spores. Surface expression of the adjuvant LTB on the surface of Bacillus U148 spores. In red, the spores were stained with a rabbit IgG isotype and in blue, with a rabbit polyclonal antibody raised against LTB.

FIG. 20 is a graphic diagram showing the results of analysis using flow cytometer for the expression of the receptor binding domain (RBD) of SARS-CoV-2 on the surface of Bacillus U50 spores. Surface expression of the RBD on sporulated Bacillus U50. In red, wild-type U50, and in blue, transformed U50 were both incubated with a RBD specific monoclonal antibody.

FIGS. 21A and 21B are graphic diagrams showing IFNγ production in response to in vitro challenge splenocytes isolated from immunized mice.

### DESCRIPTION OF EMBODIMENTS

The present technology is explained in greater detail below. This description is not intended to be a detailed catalog of all the different ways in which the technology may be implemented, or all the features that may be added to the instant technology. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure which variations and additions do not depart from the present technology. Hence, the following description is intended to illustrate some particular embodiments of the technology, and not to exhaustively specify all permutations, combinations and variations thereof.

As used herein, the singular form “a,” “an” and “the” include plural referents unless the context clearly dictates otherwise.

The recitation herein of numerical ranges by endpoints is intended to include all numbers subsumed within that range (e.g., a recitation of 1 to 5 includes 1, 1.5, 2, 2.75, 3, 3.80, 4, 4.32, and 5).

The term “about” is used herein explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value. For example, the term “about” in the context of a given value or range refers to a value or range that is within 20%, preferably within 15%, more preferably within 10%, more preferably within 9%, more preferably within 8%, more preferably within 7%, more preferably within 6%, and more preferably within 5% of the given value or range.

The expression “and/or” where used herein is to be taken as specific disclosure of each of the two specified features or components with or without the other. For example, “A and/or B” is to be taken as specific disclosure of each of (i) A, (ii) B and (iii) A and B, just as if each is set out individually herein.

As used herein, the term “Coronavirus” refers to a large group of viruses named for the crown-like spike proteins on their surface. These viruses belong to the enveloped RNA virus family Coronaviridae that animals and human share, based on the frequent mutations that lead these viruses to a rapid adaptation to one species or another. A characteristic that distinguishes these infections is a rapid spread and, often, a different pathogenicity of the viruses according to the categories, but above all to the age of the affected hosts. They consist of single-stranded positive-sense genomes and are currently classified into four genera based on the differences in their protein sequences. Genus are Alphacoronavirus, lineage A, B (as SARS-CoV) and C Betacoronavirus (as MERS-CoV), Gammacoronavirus and Deltacoronavirus (these two latter have not been reported to cause human disease). Non-limiting examples of coronaviruses include HCoV-NL63, HCoV-229E, HCoV-OC43, HKU1, SARS-CoV, MERS-CoV, and SARS-CoV-2.

In some embodiments, the present technology relates to a method for the expression of natural and synthetic antigens of SARS-CoV-2 on the external surface of a microorganism.

In some embodiments, the present technology relates to a method for the expression of natural and synthetic antigens of SARS-CoV-2 on the external surface of a member of a Bacillus genus. In some instances, the microorganism is a spore of a member of the Bacillus genus.

In some embodiments, the natural and synthetic antigens of SARS-CoV-2 includes, but are not limited to, the S protein or a portion thereof such as for example the receptor binding domain thereof, the N protein or a portion thereof such as for example the receptor binding domain thereof, or a fusion of several epitopes whose sequences are related to different SARS-CoV-2 proteins. The genetic fusion of each antigen with the coding sequences for protein adjuvants is described.

In some embodiments, the present technology relates to a method for the selection of the transformants present in the microorganism based on an approach without antibiotics.

In some embodiments, the present disclosure relates to a method for the visual detection on solid mediums of the expression of antigens on the surface of the Bacillus spores. In some instances, the present technology allows to indefinitely maintain the spores produced in a state of fermentation.

In some embodiments, the present technology describes the development of a production platform that makes it possible to display specific proteins or antigens on the surface of a microorganism, such as on the surface of Bacillus spores. In some implementations of these embodiments, the present technology relates to a method for the preparation of a vaccine in order to prevent an infection by the SARS-CoV2 coronavirus based on spores of the Bacillus genus.

In some embodiments, the system of the present technology enables the display the receptor binding protein (RBD) of the S1 protein on the surface of the Bacillus spores through a fusion between the RBD antigen and the membranal cotY protein that is expressed starting from the middle of the phase of exponential growth. A synthetic fragment expressing various peptides from different SARS-CoV-2 proteins is also described. This peptide complex (multi-epitope) has been cloned between the ATTACH protein cotY and the RBD.

In some embodiments, the present technology also relates to an expression system expressing a known protein adjuvant (LTB) on the surface of the spores. The LTB is positioned near the N-terminal of the construction. The PADRE peptide sequence (pan HLA-DR-binding epitope) was also added between cotY and LTB.

To be effective, a vaccine needs to trigger various immune cell types. B cells are an immune cell type that produces antibodies that can physically block the virus from infecting cells or reduce virus shedding. B cells generate those antibodies when they encounter tridimensional epitopes, usually from a whole protein or peptide, and this is called humoral immunity. While inducing a humoral immune response is an obvious goal in the development of COVID-19 vaccines, a strong cellular immune response is also highly desirable, considering the reports on rapidly decreasing neutralizing antibody titers in convalescent patients [4, 5], and infection-naïve vaccinated individuals [6]. Cellular immunity is mediated by T lymphocytes and is antibody independent. CD8⁺ T lymphocytes, or cytotoxic T cells, recognize unusual peptides coupled to major histocompatibility complex (MHC) class I molecules present on all nucleated cells in the body of vertebrates. The role of activated CD8⁺ in the case of a virus infection is to destroy infected cells, thereby reducing the capacity of the virus to reproduce. On the other hand, CD4⁺ T lymphocytes, or T helper cells, play a highly important supportive role in all adaptative immune responses. They recognize digested peptides bound to the MHC-II, a molecule exclusively present on the surface of phagocytic APCs. When activated, they help B cells to secrete antibodies, T cells, to kill infected cells, but also macrophages to digest ingested cell debris. It is therefore advisable for a vaccine to contain B cells, CD8⁺ and CD⁴⁺ epitopes.

As most of the current vaccines and vaccine candidates rely on the same antigen, the receptor binding domain (RBD) of the Spike protein, a chimeric peptide comprising epitopes from the highly conserved [7] nucleocapsid (N) protein, but also from the membrane (M), and ORF-1 and -3 proteins could provide a more robust and variant-resistant protective immune response. Because of the extensive research on SARS-CoV-2 and its protective immunity during the first two years of the COVID-19 pandemic, the experimental assays of the present disclosure show a collection of both dominant and immunogenic epitopes and antigens that would provide a strong, broad and durable immune response.

The virus causing SARS-CoV-2, just like other β-coronaviruses, infects cells by using a homotrimeric spike (S) glycoprotein on their envelope. SARS-CoV-2's receptor binding domain is located at the apex of the S protein and binds to the angiotensin-converting enzyme 2 (ACE2), which is expressed on the epithelium of various human organs, including the lungs, colon, liver, bladder and adrenal glands [8]. The binding of the RBD with ACE2 triggers a cascade of events that leads to the entry of the virus into the cell. The interest in using the RBD in a vaccine is the production of neutralizing antibodies. In fact, neutralizing antibodies can interfere with viral infection, and are the first line of defense against it. Injectable RBD-based vaccines can provide serum titers of neutralizing antibodies (IgG), but as SARS-CoV-2 enters the body via the mucous membranes, the induction of a mucosal immune response in the form of IgA is desirable. One of the advantages of oral vaccines is their capacity to induce the production of both IgG and IgA, so using the RBD in an oral vaccine could potentially reduce the risk of infection and contagion, not only protecting against the severe form of COVID-19.

It is also to consider that, as the vaccine immunity grows, so does the pressure on the virus to mutate to evade immune response [1]. Nevertheless, as the new variants emerge, the technology described herein is easily and rapidly adaptable by replacing the existing RBD DNA sequence vector with the new one. The modular design of the vaccine platform enables the use, if needed, of multiple variations of the RBD in the same dose, as each can be expressed on different bacterium.

To further improve the quality of immune response after immunization, a multi-epitope chimeric peptide was fused between the RBD and cotY. This peptide is comprised of antigenic epitopes selected from SARS-CoV-2 proteins that do not tend to produce highly effective neutralizing antibodies, but still can induce a strong T cell immune response. In fact, in contrast to humoral immune response, T cell immunity can target not only the structural proteins (spike (S), nucleocapsid (N), membrane (M) and envelope (E)), but also the non-structural (Nsp1-16) and accessory proteins (Orf3a, Orf3b, Orf6, Orf7a, Orf7b, Orf8, Orf9b and Orf10) [9, 10].

Based on the most recent literature, 8 epitopes from SARS-CoV2 were chosen to assemble a chimeric polypeptide, six of which were identified in convalescent patients after a 6-month longitudinal study [4, 11], and two from a list of predicted strong epitopes tested in a mouse model [12]. Also, the pan HLA-DR binding epitope (PADRE) was added to improve the construction antigenicity [13]. The peptides were then independently analyzed with bioinformatics tools to assess their potential allergenicity of toxicity (AllerTOP v2 [14], AllergenFP [5] and ToxinPred [15]), and then assessed for population coverage with IEDB population coverage tool (http://tools.iedb.org/population/). This is particularly important as the distribution of different variants of MHC-I and MHC-II molecules in the world population could, without careful consideration, greatly reduce the efficacy of the vaccine in certain ethnic groups.

A list of the peptides included in the multi-epitope is provided in table 1. PADRE, epitope number 1, is a simple carrier epitope used in the development of recombinant vaccines that can enhance adjuvant-assisted immune response [16]. Epitopes 2, 3, 4, 7, 8 and 9 were chosen because reactive CD4⁺ or CD8⁺ cells were found in the blood of convalescent COVID-19 patients [4].

All those epitopes were considered dominant, which means they were detected in over 50% of tested convalescent patients [4, 11]. Epitopes 2, 4 and 7 are cross-reactive because they were also detected in individuals never exposed to SARS-CoV-2 [4, 11], and this suggests these epitopes are highly conserved between SARS-CoV-2 and the common cold human coronaviruses (HCoVs), and could prove useful to protect against SARS-CoV-2 variants and future novel coronaviruses [17]. Epitopes 2, 3 and 4 were also identified as persistent, because they were still present 6 months after exposition [4]. No data was available for epitopes 5 to 9 in that regard. Epitopes 5 and 6 were chosen from computationally predicted epitopes that gave a strong response in a mouse model [12], and because they were predicted to bind with a large variety of MHC-II molecules, potentially improving the global coverage of the vaccine.

TABLE 1
List of peptides composing the multi-epitope

| Number | Protein | Length | Target population | Sequence |
|---|---|---|---|---|
| 1 | — | 19 | CD4⁺ | AGLFQRHGEGTKATVGEPV [13, 16] |
| 2 | N | 15 | CD4⁺ | KDGIIWVATEGALNT [4] |
| 3 | M | 15 | CD4⁺ | LSYYKLGASQRVAGD [4] |
| 4 | S | 9 | CD8⁺ | QYIKWPWYI [4] |
| 5 | M | 27 | CD8⁺ | LLQFAYANRNRFLYIIKLIFLWLLWPV [12] |
| 6 | M | 26 | CD8⁺ | FIASFRLFARTRSMWSFNPETNILLN [12] |
| 7 | ORF1 | 10 | CD8⁺ | TTDPSFLGRY [11] |
| 8 | N | 10 | CD8⁺ | KTFPPTEPKK [11] |
| 9 | ORF3 | 9 | CD8⁺ | ALSKGVHFV [11] |

Then, suitable linkers were found to assemble the peptides, according to the work of Rahmani et al., 2021 [13], Verlders et al., 2001 [18] and Yadav et al., 2020 [19]. The amino acid sequence of the multi-epitope, including the various linkers is as set forth in SEQ ID NO: 1:

(SEQ ID NO: 1)
AGLFQRHGEGTKATVGEPVGGGSKDGIIWVATEGALNTAAYLSYYKLGA
SQRVAGDHEYGAEALERAGQYIKWPWYIAAYLLQFAYANRNRFLYIIKL
IFLWLLWPVAAYFIASFRLFARTRSMWSFNPETNILLNAAYNSASFSTF
KCYGVSPTKLNDLCFTNVAAYTTDPSFLGRYAAYKTFPPTEPKKAAYAL
SKGVHFV,

wherein underlined are the various epitopes, in order from 1 to 9, as presented in Table 1. In italics and in bold is a flexible linker, in gray shade is the linker that separates MHC-I and MHC-II binding epitopes and the AAY linker helping with epitope processing. The AAY spacer was used between epitopes because it is believed to improve epitope processing [18]. Then, the HEYGAEALERAG linker was used to separate MHC-I and MHC-II binding epitopes [13, 20]. Moreover, a GGGS flexible linker was used to separate the PADRE epitope from the MHC-II binding epitopes.

Also, to induce an appropriate immune response, vaccines need adjuvants. The definition of adjuvant is a pharmacological or immunological agent capable of increasing, modulating or prolonging an immune response towards an administered antigen. They can either activate the innate immune system by triggering pattern-recognition receptors (PRRs), or concentrate and display antigens in repetitive patterns, so they are efficiently presented to antigen-presenting cells (APCs) [21-23], both of which are non-exclusive.

Among the multiple possible “new” adjuvants, E. coli heat-labile enterotoxin B chain (LTB) is of great interest, because contrarily to most market-approved adjuvants, it is compatible with a wide range of antigens, and can be useful in both parenteral and mucosal vaccines [23, 24]. LTB is a subunit of the Escherichia coli heat-labile toxin (LT). LT consists of 5 LTB domains arranged around a catalytic domain (LTA), which possesses a strong ADP-ribosyltransferase activity [25, 26]. LT is a member of the AB5 bacterial toxin family and is closely related to the cholera toxin of Vibrio cholerae [23]. The nontoxic beta subunit (LTB) is a well-known mucosal immunogen [25], safe in humans [27], and was used both as a free adjuvant and fused with antigens [28-33]. Although LTB could exert its adjuvanticity by binding the GM1 ganglioside receptor, thereby affecting the turnover, development and antigen presentation of dendritic cells, the clustering of lymphocytes, and the B cell uptaking of antigens [24, 25], its mechanism of action remains unclear [34]. Still, Lee et al. reported that LTB upregulates the transcription of TNFα, IL-12, IL-1αβ, IL-2 IL-6 and chemokines CXCL1, CXCL2, CXCL3, CCL2, CCL3, CCL4 and CXCL16 in dendritic cells (DCs) and these changes in DCs are likely to be critical in triggering a strong immune response [35].

In the design detailed in the current t, the LTB is attached to a different spore than that of the antigen, and since the dose is potentially critical to the adjuvant effect [23], this feature allows the fine-tuning of the dosage of both the antigen and the LTB adjuvant. Also, the PADRE epitope, comprised in the multi-epitope, is considered an adjuvant, as it binds to almost all variants of HLA-DR and, thus, activates CD4⁺ cells. In fact, as cytokines produced by CD4⁺ cells are of paramount importance in the body's defense against viruses, the use of a universally compatible CD4⁺ epitope ensures a CD4⁺ response.

The PADRE epitope, in the current technology, is present on the surface of both LTB- and RBD-bearing spores. Furthermore, bacilli from the Bacillus subtilis genus, by their surface expression of peptidoglycan, have an intrinsic adjuvant effect. It was recently described that both peptidoglycan alone and bacilli spores can induce the production of TNFα, IL-1β, IL-6 and IL-10 by cultured DCs differentiated in vitro from human peripheral blood mononuclear cells (PBMC) [36]. The chosen design is also highly relevant because it concentrates the antigens on the surface of aparticle (spore), which is also considered as an adjuvant strategy in and of itself. In consequence, the combination of antigen-bearing spores with LTB-bearing spores in one vaccine should, in theory, be sufficient to induce a strong mucosal immune response.

This complex and modular design of adjuvants leaves the possibility to assemble different combinations of the vaccine, with and without LTB-bearing spores, for example, to achieve the best immune response possible in every target population.

A protein coding for the phospholipase C activity of Bacillus cereus is associated by fusion to the membrane spore protein cotY. This function, naturally absent from Bacillus strains not belonging to the species Bacillus cereus, Bacillus thuringiensis and Bacillus anthracis, makes it possible to quickly detect on a solid medium the presence or absence of the fusion of proteins displayed on the surface of a Bacillus spore. The phospholipase activity can be assayed by spectrophotometry in order to select the best transformants. Once the clones of interest have been selected, the coding gene for the phospholipase C is no longer necessary and then eliminated by PCR.

The selection of transformants is carried out without the use of an antibiotic-resistance gene present in E. Coli as well as B. subtilis. The selection of transformants is carried out on a minimal medium with sucrose present in E. Coli. In order to do so, the coding gene for the cscA protein (sucrose-6-phosphate hydrolyse) was cloned in the different coding vectors for the antigens of interest. Since the E. coli laboratory strains are incapable of growing in a minimal medium with added sucrose, the addition of the coding gene for the CscA protein makes it possible to select the transformants present in E. coli. In the case of Bacillus Subtilis, this bacterial species is incapable of growing in a minimal medium with added inulin. However, the addition of a coding gene for an inulinase has allowed the selection without difficulty of transformants thanks to the addition of this new genotype that is naturally absent in the wild strain of interest.

With the aim of creating a vaccination system that is safe for man and for the environment, a strain of B. Subtilis cannot germinate was developed. To do so, the sleB gene was knocked-out of B. subtilis U50. That gene codes for a enzyme implicated in the germination process of spores into vegetative cells. The vaccine produced in this way will always remain in the form of spores. This will have the advantage of preventing the active growth of vegetative cells that are active in the digestive system. In addition, despite using a bacterial strain usually known to be GRAS (Generally Recognized As Safe) in which foreign DNA that does not contain coding genes for resistance to antibiotics is introduced (plasmid or chromosome integration), the non-germination property makes it possible to avoid the dispersion of genetically modified cells into nature.

The vaccinal platform of the present technology may be used to produce a Bacillus-spore based vaccine. In some instances, the Bacillus-spore based vaccine of the present technology may be administered orally or nasally or intramuscularly. Once at the mucosa, the antigens attached to the surface of the Bacillus spores will come into contact with the dendritic cells of the intestinal mucosa which stimulates the immune system of the host.

Display technology was first achieved using peptides that were fused to phage surface proteins (Scott and Smith 1990). Little et al. (1993) and Georgiou et al. (1993) were pioneers in describing functional display systems in Gram-negative bacteria [37,38]. The first functional display systems in Gram positives were described in 1995 [39-41]. As regards eukaryotes, the organism most used to achieve protein cell display is without a doubt the yeast Saccharomyces cerevisiae. Schreuder et al. (1993) used a display system based on the use of alpha agglutin as a natural anchor protein for the first time [42]. Since that time, a large number of proteins coding for enzymatic activities of interest have been displayed on different living organisms. The use of such systems quickly appeared to be an interesting alternative with regard to the idea of producing microorganisms in fermentation on which are immobilized a large number of enzymes which are both active and whose cells are easy to recover in order to be able to use the displayed catalysts again.

The protein cell display technology was then quickly applied in the field of human and animal health. One of the ideas was to explore the possibility of using non-pathogenic living organisms as an antigen-presenting vector. The organism that produces a large amount of a specific antigen on its surface can then be absorbed orally or nasally to induce the immune system against the displayed antigen. At the veterinary and medical level, there are more and more examples in the literature demonstrating the great potential for using protein cell display technology. For example, Kang et al. (2021) developed an oral administration system of glucagon-like peptide 1 (GLP-1) (28-36) for the treatment of type 2 diabetes by achieving the quintuple peptide 1 display (28-36) of the glucagon type on Bacillus subtilis spores[43]. For their part, the team of Oh et al. (2020) carried out the display on spores of B. subtilis of the protective agent which is one of the three virulence factors associated with anthrax [44]. Immunization by oral or nasal administration was able to induce the immune system of treated mice by conferring active immunity in a mouse model based on the results of antibody isotype titration, and identification of mucosal antibodies. Kang et al. (2020) succeeded in displaying the P75 protein, a Lactobacillus rhamnosus protein, on the surface of B. subtilis spores [45]. The spore and protein complex P75 demonstrated its effectiveness in partially degrading the wall of Listeria monocytogenesis which is a recognized foodborne pathogen. Regarding the veterinary sector, Li et al. (2020) successfully displayed the porcine rotavirus VP8 antigen [46]. The oral administration of this live vaccine allowed the detection of a strong specific mucosal and humoral immune response, which would make this system a simple, effective and inexpensive alternative for the prevention of porcine rotavirus. Cen et al. (2021) developed a molecular attachment system that displays different antigens on the surface of Saccharomyces cerevisiae, which have made it possible to successfully produce oral vaccines to prevent Helicobacter pylori infections [47]. This bacterium causes a major gastric infection when it settles in the digestive system. Regarding S. cerevisiae, Lei et al. (2020) presented results which demonstrate that the display of the HA (hemagglutinin) antigen of the H5N1 virus on the surface of this yeast makes it possible to effectively immunize mice thanks to the production of neutralizing antibodies following oral absorption of the developed vaccine [48].

The concept of live vaccines is initially based on the use of microorganisms of the probiotic type recognized for their use in food for millennia. The most studied probiotics belong to the Saccharomyces and Lactobacillus genus. The probiotic Bacillus subtilis, as well as strongly related strains, are increasingly becoming a very interesting alternative. These three living organisms are known for being genuslly recognized as safe (GRAS), which considerably facilitates their acceptability at the regulatory level.

In the case of the yeast Saccharomyces cerevisiae and lactic acid bacteria belonging particularly to the genus Lactobacillus, it has long been recognized that, like the genus Bacillus, these organisms are considered to be GRAS. There are also a multitude of products on the market in which one or the other of these organisms is found. The latter have the advantage of being easily produced in large-volume fermentation. Their recovery is usually done by centrifugation but also by tangential filtration. The concentrate of these organisms can then be transformed into different forms.

Despite the fact that the preparation of yeasts of the Saccharomyces cerevisiae type and bacteria of the Lactobacillus type require a more complex harvesting step than that of the genus Bacillus, the use of these two organisms as a live vaccine remains an interesting alternative. Like Bacillus, it is easy to obtain a finished product in dry form whose level of viability will meet the regulatory requirements for obtaining a live vaccine that is stable over a long period (6 to 12 months) at room temperature. The importance of the cold chain does not apply to a product in dry form.

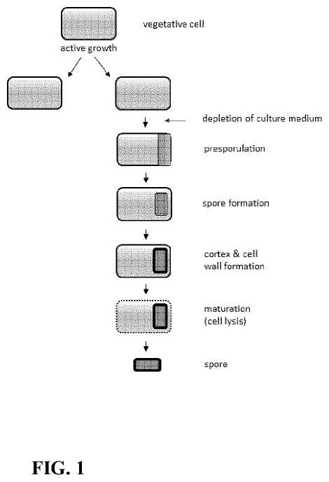

For the formulation of a live vaccine in liquid form, it is also possible to use Lactobacillus in drinking yogurts, which will not be the case for yeasts. This type of product may find its market in industrialized countries but unfortunately, not in hot countries with a low GDP. Despite the possibility of being able to formulate vaccines in liquid form with lactic acid bacteria, the shelf life is not long enough to consider such an approach because the logistics of distribution in hot and developing countries would not make it possible to offer a product quickly enough. This problem is similar for Saccharomyces-based fermented products. However, it is possible to dry these two microorganisms and to use them in the form of an oral tablet. Although this approach is very interesting, it must also be considered that transformation costs are also very high, which could eventually reduce the level of accessibility of this type of vaccine for developing countries. In addition, the dry form eliminates de facto the possibility to develop a vaccine that can be administered nasally. These two drawbacks are not present for vaccines based on the use of Bacillus spores. FIG. 1 shows the principal use of the Bacillus spore vaccine display.

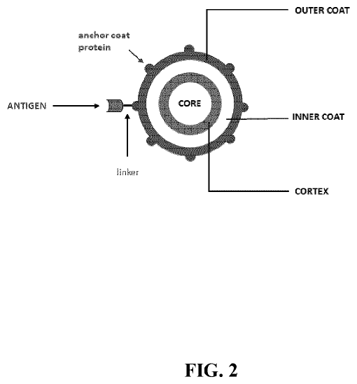

Of these three organisms, the genus Bacillus is the only one that has the quality of being able to sporulate. Being able to pass from the vegetative state to the spore state gives Bacillus the advantage of obtaining a bacterium which can be preserved very easily over a period varying from a few months to a few years, both in the form of a dried and liquid product. This evolutionary advantage allows the genus Bacillus to be used in n highly stable form, which no other organism in the world of microorganisms possesses. FIG. 2 shows the genus Bacillus sporulation process.

When properly prepared, Bacillus spores can be stored at room temperature for years. The need to respect the principle of the cold chain then becomes unnecessary. Bacillus spores are extremely resistant to pH variations. This characteristic has the advantage that probiotics based on Bacillus spores easily resist the conditions encountered in the digestive system.

In some embodiments of the present technology, the following Bacillus may be used: Bacillus subtilis, Bacillus circulans, Bacillus clausii, Bacillus amyloliquefaciens and Bacillus velezensis.

The production of spores in fermentation depends on, without being limited to, the composition of the culture medium, on the pH, the temperature, the concentration of trace salts, the stirring rate and the concentration of dissolved oxygen in the medium. More specifically, sporulation is induced by certain minerals such as cobalt salts, manganese salts, magnesium salts, calcium salts. The carbon-to-nitrogen ratio influences both the quantity of bacteria as well as the rate of sporulation of the genus Bacillus. Some bacilli will have a high sporulation rate when the dissolved oxygen concentration is high while others will be unable to sporulate at this concentration. The stirring rate greatly influences the sporulation rate because stirring directly influences the level of dissolved oxygen present in the culture medium. The addition of a feed also has a very significant impact on the final count as well as on the level of sporulation. The carbon-to-nitrogen ratio as well as the presence of trace mineral salts will again have a major effect on the final sporulation rate. A rate between about 70 and about 99%, or between about 85 and about 99%, or between about 95 and about 99%, is sought.

One of the great advantages of developing a live Bacillus spore vaccine is that it is easy to obtain a spore powder either by freeze-drying or by spray dryer, just as it is simple to produce a finished product in very stable liquid form. In the case of freeze-drying, it is usually necessary to add one or more agents to the preparation to be dried that will minimize as much as possible the loss of viability of the microorganisms caused by the drying method used. Bacilli are much less subject to the instability often encountered during the freeze-drying process, due to the reinforced structure of the spore. To minimize the potentially negative impacts, the producer will only have to add ingredients known for their cryoprotective effects. The main cryoprotective agents used are maltodextrins, skimmed milk, dextran and sucrose. In the case of the spray dryer, fine-tuning of the vaporization rate and the drying temperature must be taken into consideration because these parameters are critical for maintaining viability. With respect to a live vaccine containing Bacillus spores, the structure of the spore provides a very high level of long-term stability which is impossible to obtain for both vegetative bacteria and yeasts. The possibility of obtaining a live Bacillus spore vaccine can make it possible to produce drinkable formats, which is very advantageous for the oral vaccination of children. Adding spores of a Bacillus vaccine to a drinking beverage is an example of a format that is simple and easy to produce and is very appealing to children. This type of product will find all its usefulness in large-scale distribution in developing countries as well as in countries with hot climates.

Being able to obtain spores in the laboratory as well as in large-scale fermentation allows for the creation of several types of finished products with extremely long shelf lives. In agriculture, there are finished products based on Bacillus spores which are used to promote plant growth or to protect them against various fungal or bacterial diseases. The spores can germinate within a few hours in order to perform the function for which they were selected. There are also bacterial products made from Bacillus spores that are commonly used in wastewater treatment.

More recently, Bacillus spores were used as a support for the adsorption of heterologous proteins (enzymes, antigens) in order to significantly increase their half-life [49] but also to allow their reuse when these proteins are used as a catalyst [50].

An example of the use of spores as an adsorption agent for antigens was carried out by Huang et al. (2010) when the latter achieved, inter alia, the adsorption of the fragment C of the tetanus toxin antigen [51]. This approach is very different because, on the one hand, it is necessary to produce Bacillus spores and, on the other hand, in another different fermentation, to produce the antigen of interest. Although interesting, this approach greatly complicates the display process and significantly increases production costs, making such a solution much less attractive from an economic point of view.

One of the first uses of Bacillus spores as a display system (spore display) for an enzyme through a transmembrane anchor protein expressed during the sporulation phase was carried out in the early 2000s. Isticato et al. (2001) fused the C fragment of tetanus toxin to the membrane protein cot B [52]. Several other examples in the scientific literature describe the use of the Bacillus spore display system in order to express enzymes attached to the surface of these same spores [53-57].

In turn, U.S. Pat. No. 7,582,426 [58] as well as U.S. Pat. No. 20040171065 [59] disclose a method which makes it possible to display a protein of interest on the surface of a spore of Bacillus subtilis. This method presents different display examples using either the cotE and cotG anchor proteins present in the spore. Obtaining the functional activity of a B galactosidase as well as a cellulase displayed on the surface of B. subtilis spores has also been demonstrated. An objective of the technology proposed here is to display new functionalities on the surface of a B. subtilis spore that are naturally absent in the wild strain.

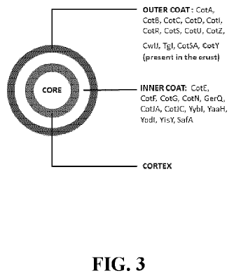

There are several existing display systems based on the use of an anchor protein present on the surface of a spore of the genus Bacillus. The most commonly used anchor proteins are associated with the cot family. FIG. 3 shows the positioning of the main cot proteins in Bacillus spores. Cot proteins that may be used in the technology are cotA, cotB, cotC, cotD, cotE, cotF, cotG, cotH, cotK, cotL, cotM, cotS, cotT, cotV, cotW, cotX, cotY, cotZ, cotJA, cotJC. In some instances, the cot protein used in the present technology is selected from cotB, cotE, cotX, cotY, and cotZ. In some instances, the cot protein used in the present technology is cotY. There are other anchoring proteins including, for example, SpoIVA, SpoVID or even SodA (WO 02/46388) [60]. Some of these anchor proteins (cotA, cotB, cotC, cotD and cotF) were used to display antigenic proteins on the surface of B. subtilis, such as the SLT-1B subunit of Clostridium perfringens (U.S. Pat. No. 5,800,821) [61]. The results obtained made it possible to demonstrate that the expression of the SLT-I B antigen on the surface of genetically modified B. subtilis can be obtained. No trial evaluating the production of antibodies following oral or nasal absorption in an animal model has been presented.

Flick-Smith et al. (2012) (EP1858550) presented results on the use of the Bacillus anthracis cotN anchor protein to which a recombinant protective antigen of B. anthracis is fused, all on the surface of a Bacillus spore. The intranasal use of the spore vaccine in mice induces the production of antibodies against the antigen (WO 2006/095176, U.S. Pat. No. 8,105,613) [62]. However, the administration of the vaccine has not been evaluated following oral absorption in order to assess the mucosal immune response associated with the stimulation of dendritic cells present in the intestinal wall.

In turn, U.S. Pat. No. 9,845,342 discloses different protein spore display systems developed for Bacillus cereus [63]. These systems consist of attaching different proteins to the surface of the spores of this species which provide the genetically modified strain with new properties. When a solution of these new spores is applied to seeds, the new characteristics displayed on the modified spores biostimulate plant growth.

U.S. Pat. No. 2002/0150594 [64] and No. 20030165538 [65] disclose that the addition of a linker, whose coding sequence corresponds to the HA11 epitope, makes it possible to detect, using anti HA11 antibodies, whether the molecular construct is expressed on the surface of the spores. Confirmation of the presence of a protein cell display construct is always used when the protein expressed on the surface of the spores has no detectable enzymatic activity. The use of flow cytometry is widespread in order to quickly determine the presence of a new protein on the surface of a spore or a vegetative cell using an antibody specific to the protein expressed compared to the non-genetically modified wild strain. It is important to emphasize that there is no system in the literature for detecting a protein or antigen having no measurable functional activity coupled with a protein having an easily measurable enzymatic activity that is naturally absent in the genetically modified body.

In turn, U.S. Pat. No. 20090098164 [66] discloses that the use of Bacillus cotB1 and cotB2 membrane spore proteins provide a good tethering system when enzymes or antigens are fused to them. In addition, they present results demonstrating the production of antibodies against fragment C of tetanus toxin when mice absorbed genetically adapted spores either orally, nasally, intramuscularly or intravenously. Tetanus toxin is separated from the cotB1 or cotB2 attachment proteins by, inter alia, a linker whose amino acid sequence corresponds to the cleavage zone of a protease. The system is known to work in different bacilli, specifically in B. subtilis, B. amyloliquefaciens, B. anthracis, B. thuringensis, B. cereus, B. weihenstephanensis, Geobacillus kaustophilus and Geobacillus thermodenitrificans. A relatively similar approach is also disclosed in U.S. 2011/0104200 [67]. The use of the membrane protein cotC, present at the time of sporulation, was selected as a binding model for different antigens. The use of a rigid linker between the attachment protein and the antigen of interest is not disclosed. Such a linker has the advantage of moving the antigen away from the surface of the spore and increasing its exposure, thereby maximizing contact between the antigen and the dendritic cells present in the mucous membranes. Several viral targets are mentioned, but none related to a coronavirus.

All systems using protein cell display technology are obtained through different cloning and genetic modifications. The construction of a platform of this type always begins in a laboratory strain of the Escherichia coli type. E. coli has been used for several decades because this bacterium is easy to manipulate at the molecular level, it grows quickly and most of the molecular tools have been developed in order to use them in this bacterium. It is widely recognized that the cloning steps involve the introduction of a DNA fragment into a plasmid and, to a lesser extent, into a cosmid or a fosmid. The fragment must then be introduced by means of different methods into a plasmid which will be linearized either following enzymatic digestion or following PCR amplification of the area of interest which includes, minimally, the origin of replication of the plasmid so can it can divide in E. coli and a gene coding for resistance to an antibiotic. The presence of this gene is essential because it is thanks to the latter that the cells of E. coli, which were efficiently transformed with the plasmid in which a gene of interest was cloned, can be selected. The principle of antibiotic selection is also used in other microorganisms in which a circular or linear molecular construct (for chromosomal integration) must be introduced.

There are bifunctional systems which allow, with a single plasmid, manipulation of the latter by introducing DNA fragments of interest, transforming E. coli in order to produce plasmid DNA which will then be used to transform another bacterial or yeast strain which will be the final recipient organism. Again, it is usually accepted that the use of an antibiotic-resistant gene is necessary in order to be able to select the genetically transformed recipient organism.

However, the use of a selection system based on the presence of a gene coding for resistance to an antibiotic is not compatible for applications in which a higher organism (plants, animals, humans) will be genetically modified. This leads to an insurmountable health, ethical and regulatory problem. In order to solve this serious problem, it is necessary to develop cloning systems whose selection system is based on an alternative approach. There are precedents in the literature. For example, in Saccharomyces cerevisiae, Liu et al. (2017) developed a selection system based on the addition of the SSU1 gene which allows a transforming strain to grow on a sulfite-based selection medium [68]. Without the SSU1 gene, a wild strain of S. cerevisiae has a very low tolerance to the presence of sulfite. This tolerance is multiplied by 4 in the presence of the SSU1 gene. In Escherichia coli, several auxotrophic mutants were developed. It is then relatively easy to be able to complete the auxotrophy by introducing a gene coding for the function to be complemented. Furthermore, it is known that the laboratory strain E. coli K12 and its derivatives are not capable of growing in the presence of sucrose. This characteristic can become an advantage because the addition to a plasmid of the cscA gene, which codes for a sucrose hydrolase, makes it possible to confer the ability to strains of E. coli to grow on minimal medium supplemented with sucrose [69].

In Bacillus, counter-selection systems are much rarer. Champney and Jensen (1969) demonstrated that B. subtilis 168 is intolerant of the presence of D-tyrosine[70]. For their part, Geraskina et al. (2015) demonstrated that unlike B. subtilis 168, the B. amyloliquefaciens A50 strain is resistant to the presence of the D-tyrosine isomer [71]. They were able to demonstrate that it is the yrvI gene, which codes for a d-tyrosyl-tRNATyr deacylase, which makes the A50 strain resistant to the presence of D-tyrosine. When they clone this gene into a plasmid and transform B. subtilis 168, the latter then becomes capable of growing in the presence of D-tyrosine, which confirms the relevance of using the yrvI gene as a selection gene in strains of D-tyrosine sensitive bacilli.

A complete review of the disclosures made in the various patent databases made it possible to draw attention to the fact that, at this time, there is no platform describing a protein cell display system using plasmid constructs using counter-selection genes free of antibiotic-resistant genes. This finding is surprising because counter-selection based on technologies without antibiotic-resistant genes is essential. Alternatives must be put forward so that a technology intended to be used at the animal and human level may have a chance to be marketed in the respect of the different international regulatory organizations. In addition, an exhaustive review of the scientific literature has also made it possible to detect that, at this time, no E. coli-Bacillus bifunctional plasmid exists in which the screening of the transformants present in E. coli and Bacillus can be carried out without the presence of a selection system without an antibiotic-resistance gene.

In one embodiment, the present technology relates to a method for producing spores of the genus Bacillus on which molecular modifications offer the advantage of exposing on the surface of said spores a new function which may be of the enzyme, antigen, receptor, antibody or functional protein type.

As used herein, the expression “molecular modification” refers to any modification made to different DNA fragments manipulated by humans. These modifications includes, without being limited thereto, the amplification by PCR (polymerase chain reaction) of specific DNA fragments using short single-stranded DNA primers obtained by chemical synthesis. These primers are homologous to a very precise zone which is defined by the manipulator. The chemical synthesis of long double-stranded DNA fragments, always defined by the manipulator, is part of the molecular modifications. DNA fragments of interest, lying between two restriction enzyme sites, may also be used. These fragments obtained following digestion, the synthetic DNA fragments or the PCR products may subsequently be cloned into a plasmid vector at a well-defined location in this same plasmid. The DNA fragments of interest may contain regulatory zones such as a promoter, aribosome binding site, the coding sequence of a gene of interest starting with a start codon and ending with a termination codon. The receptor plasmid may, in turn, contain a transcription terminator located very close to the termination codon. Without being limited thereto, the recipient plasmid must contain at least one or two origins of replication in order to facilitate the replication of said plasmids in E. coli and possibly in a second host. According to the need, it will be necessary to find, on one or two genes encoding enzymes which enable the selection of transformants on solid mediums with the addition of a specific ingredient. Selection will usually be done using antibiotics for laboratory manipulations and non-toxic non-antibiotic chemical agents for manipulations aimed specifically at animals and humans. All of these “molecular modifications” will aim to create Bacillus spores on which functional proteins, enzymes, antigens or antibodies will be displayed.

The genetically modified Bacillus spores developed in this platform are not limited to B. subtilis. These modifications also target the B. amyloliquefaciens group which includes B. amyloliquefaciens, B. velezensis, B. methylotrophicus and B. siamensis. B. subtilis strains including all subspecies are also included. Bacillus circulans spores can also be transformed with the molecular system presented in the disclosure.

In order to favor the expression of the gene fusion so that the translated proteins can be displayed at the external part of the spores, the promoter selected may be, without being limited thereto, PcotYZ, pCotVWX, PcotX, PcqeA, PcgeA, PcgeAB or PcotE.

The attachment proteins included in the platform may come from structural proteins of the cot family, which includes, without being limited thereto, cotA, cotB, cotC, cotD, cot E, cotF, cotG, cotM, cotN, cotS, cotT, cotV, cotX, cotY, cotZ, cotJa, cotJC but also the membrane spore proteins SpoIVA, SpoIVD and safA.

As used herein, the term “linker” is defined as a sequence of amino acids whose function is to separate two peptides, a peptide from a protein, or two proteins from each other. In addition to separating two distinct regions from each other, the applicant may also add a cleavage site for a protease in order to eliminate, if necessary, the region located at the carboxy terminal of the protein, enzyme, antigen or antibody located immediately upstream of the cleavage site. The linker may also be defined as being “rigid” or “flexible”. Two types of amino acid sequences used in the present technology are: rigid linker: EAAAKEAAAKEAAAK (SEQ ID NO: 2); Flexible linker: GGGGSGGGGSGGGGSGGGGS (SEQ ID NO: 3).

As used herein, the term “protein” defines any amino acid sequence which may or may not have a functional activity.

As used herein, the term “enzyme” defines any protein comprising essentially a lyase, reductase, hydrolase function or any other biological function of a catalytic nature. In the context of the present technology, the enzymatic activities sought are, without being limited thereto, lipase activity (hydrolysis of lipids), phospholipase activity (hydrolysis of phospholipids), protease activity (hydrolysis of proteins), peptidase activity (hydrolysis of peptides), cellulase activity (hydrolysis of cellulose), xylanase activity (hydrolysis of xylan), pectinase activity (hydrolysis of pectin), pectate lyase activity (hydrolysis of pectate), amylase activity (hydrolysis of starch), urease activity (hydrolysis of urea), arabinofuranosidase activity (hydrolysis of arabinofuranoside), chitinase activity (hydrolysis of chitin), chitosanase activity (hydrolysis of chitosan), hemicellulase activity (hydrolysis of hemicellulose), keratinase activity (hydrolysis of keratin), inulinase activity (hydrolysis of inulin, mannanase activity (hydrolysis of mannan), ligninase activity (hydrolysis of lignin), laccase activity (hydrolysis of aromatic compounds), lytic polysaccharide monooxygenase activity (hydrolysis of chitin), phytase activity (hydrolysis of phytate) and any other hydrolase-like activity.

In one aspect of the present technology, a gene whose function is to code for a hydrolase activity is added on the surface of Bacillus spores. In order to confirm the presence of a protein of interest on the surface of a cell, the use of an antibody is usually recommended. Despite the need to use this method, the detection of an enzymatic activity, naturally absent in the wild-type strain, makes it possible to know much more quickly and easily whether the expression system has worked well. Another advantage of the technology is that the detection on solid medium of a new functional activity makes it possible to very quickly select the best candidates transformed with the molecular construct. The best transformants are subsequently placed in liquid culture in order to allow the various Bacillus clones to grow in a medium promoting sporulation. The spores obtained are recovered, washed and their quantity standardized. The enzymatic activity expressed on the surface can then easily be quantified using an appropriate test. The higher the enzymatic activity of a transformant, the greater the display on the surface of the spore should be.

In one embodiment, the technology includes the step of introducing a rigid linker between the membrane attachment protein and the amino acid sequence coding for an enzyme whose functionality is naturally absent in the non-genetically modified Bacillus strain. The presence of the rigid linker allows to reduce steric hindrance that may exist between the carboxy terminal end of the attachment protein and the enzyme. This may assist in preserving a three-dimensional structure making it possible to preserve the functionality of said enzyme. The rigid linker may also allow to move the enzyme away from the exosporium, which promotes a maximum level of display of the enzyme. The (EAAAK)n sequence is one of the most commonly used linkers when rigidity is sought. The following rigid linkers are also contemplated: (EP)n, (KP)n, (AP)n, and (TRP)n.

Immediately following the coding sequence for the display sensing enzyme expression is a second flexible linker that separates the enzyme from the protein, antigen, sensor protein or antibody of interest. The flexibility of the linker promotes a three-dimensional movement of the amino acid sequence of interest. This characteristic is particularly useful for the exposure of an antigen or a receptor which will then be more ready to come into contact with their corresponding functional unit at the cellular level. The (GGGGS)n, (GS)n and (G)n sequences are usually the most commonly used. Chen et al., (2013) summarize the advantages and the importance of using linkers between two protein domains [72].

In some implementations, the multi-epitope design including: GPGPG, AAY, and HEYGAEALERAG linkers were used to promote epitope priming [73-75]. Indeed, numerous multi-epitope vaccines designs have been used [75-79], because they make it possible not only to avoid the creation of neo-epitopes formed at the junction between two epitopes of interest [75], but they also favor cleavage at these places through proteasome and immunoproteasome, improving the presentation of antigens through the MHC-I and the MHC-II [18, 80, 81].

In another aspect of the present technology, the platform for cloning and expressing proteins of interest to be displayed on the surface of spores of the genus Bacillus is based on a selection system without antibiotics. This aspect entails that the spores can be used both in animals and humans. The different plasmids obtained have two distinct origins of replication, the first origin allowing replication in E. coli, the second origin allowing the episomal replication of the plasmid when the latter is transformed into the genus Bacillus. The origin of replication for E. coli that is used is pMB1 plasmid derivative that is the ancestor of pUC19. For Bacillus, there are several origins of replication that are commonly used. The most common are based on replication in rolling circle mode. A large number of commercial plasmids are based on Staphyloccocus aureus repB whose basic sequence comes from the pUB110 plasmid [82-84]. Plasmids whose origin of replication is based on repB are reputed to normally be functional in Bacillus. The repU origin of replication deriving from the Bacillus cereus pBC16 plasmid is in turn a close cousin to S. aureus repB [85].

The genes coding for proteins of interest may be cloned in the platform described in the present technology. Specific examples of surface display of Bacillus spores include, but are not limited to: display of enzymatic activity useful to catalyze a reaction in order to create a bioproduct of interest; display of an enzymatic activity useful in the biostimulation of plant growth when said spores are applied to the soil at watering time; display of an enzymatic activity useful in the bioprotection of plants (chitinase, chitosanase); display of a fluorescing protein or chromoprotein useful for studying root biofilm formation or introducing the genetically modified Bacillus strain into a plant as an endophyte; display of an antigen of interest in order to produce a live vaccine based on spores of the genus Bacillus.

The present technology has been designed to produce Bacillus spores stable to extreme temperature and pH variations, resistant to desiccation, stable over a very long period when used in a liquid product, easy to produce at very large volumes, easy to sporulate after 48-96 hours of culture, easy to recover, easy to wash after recovery and easy to stabilize no matter the shape. Due to the fact that the selection of transformants is done using genes coding for antibiotic-free selection systems and that the strains of Bacillus used are free of genes coding for toxic proteins, the spores of genetically modified Bacillus are reputed to be GRAS.

The present technology allows for the possibility to create “eternal” spores. When the applications targeting the use of the spore display platform are governed by severe regulatory aspects, it is therefore advisable to avoid that the spores obtained after fermentation are able to germinate and form active vegetative cells. Examples of applications requiring the use of “eternal spores” are, without being limited to: the development of human vaccines, animal vaccines, the use of spores in the preparation of ingredients used in the food sector, or the application of spores in the environment. Several genes are implicated in the germination process for Bacillus spores. The main genes that have a non-negligeable impact on germination rates are the following: gerD, cwID, cwIJ, and sleB. The last two coding genes for lytic enzymes specific to the germination process of spores in vegetative cells [86, 87]. It has been shown that the creation of a coding gene knock-out for the SleB protein makes it possible to obtain a rate of effectiveness of the non-germination of Bacillus spores of more than 99.9%. An equivalent level of effectiveness is also obtained by creating a mutant cwIJ [86]. For their part, Sayer and Popham (2019) demonstrated in Bacillus anthracis that the presence of the YpeB protein was essential for the good working order of the SleB lytic enzyme [88]. In their case, Sun et al., (2020) in turn developed a mutant strain of B. subtilis for SleB and cwIJ genes by introducing, through dual integration, a molecular construct enabling the display the Vp7 antigen spore [89]. The expression of Vp7 on the spores' surface made it possible to obtain a good level of protection against reovirus in herbivorous carps. The integration at the SleB and vwIJ gene level blocked spore germination in the carp's digestive system. This offers the advantage of maintaining optimal quality for the Vp7 antigen without being degraded by the digestive system at the time of spore germination. It is therefore of interest to produce eternal spores in order to preserve a maximum quantity of antigens on their surface when the vaccine is absorbed, particularly by mouth. Otherwise, the antigens will be freed of the spores at the time of germination to then be quickly hydrolyzed in the digestive system, without having had the time to be in contact with the dendritic cells.

According to one embodiment of the present technology, the production of spores exhibiting proteins of interest on their surfaces may be produced in any type of fermenter, as long as the culture conditions comply with all the rules leading to sterile production.

According to some embodiments, the present technology relates to composition and vaccines. Compositions and vaccines of the present technology may be administered by any suitable means, for example, orally, such as in the form of pills, tablets, capsules, granules or powders; sublingually; buccally; parenterally, such as by subcutaneous, intravenous, intramuscular, intraperitoneal or intrastemal injection or using infusion techniques (e.g., as sterile injectable aqueous or non-aqueous solutions or suspensions); nasally, such as by inhalation spray, aerosol, mist, or nebulizer; topically, such as in the form of a cream, ointment, salve, powder, or gel; transdermally, such as in the form of a patch; transmucosally; or rectally, such as in the form of suppositories. The present compositions may also be administered in a form suitable for immediate release or extended release. Immediate release or extended release may be achieved by the use of suitable pharmaceutical compositions, or, particularly in the case of extended release, by the use of devices such as subcutaneous implants or osmotic pumps.

It is often advantageous to formulate oral or parenteral compositions in dosage unit form for ease of administration and uniformity of dosage. Dosage unit form refers to physically discrete units suited as unitary dosages for the subject to be treated; each unit containing a predetermined quantity of active compound calculated to produce the desired therapeutic or immunogenic effect in association with the required pharmaceutical carrier.

In an embodiment, a composition or vaccine is prepared as an injectable, either as a liquid solution or suspension, or as a solid form which is suitable for solution or suspension in a liquid vehicle prior to injection. In another embodiment, a composition or vaccine is prepared in solid form, emulsified or encapsulated in a liposome vehicle or other particulate carrier used for sustained delivery. For example, a vaccine can be in the form of an oil emulsion, a water in oil emulsion, a water-in-oil-in-water emulsion, a site-specific emulsion, a long-residence emulsion, a sticky emulsion, a microemulsion, a nanoemulsion, a liposome, a microparticle, a microsphere, a nanosphere, or a nanoparticle. A vaccine may include a swellable polymer such as a hydrogel, a resorbable polymer such as collagen, or certain polyacids or polyesters such as those used to make resorbable sutures, that allow for sustained release of a vaccine.

In some embodiments, compositions provided herein include one or more additional therapeutic or prophylactic agents for coronavirus infection. For example, a composition may contain a second agent for preventing or treating coronavirus infection. Examples of such second agents include, without limitation, antiviral agents.

In alternative embodiments, compositions of the present technology may be employed alone, or in combination with other suitable agents useful in the prevention or treatment of coronavirus infection. In some embodiments compositions of the present technology are administered concomitantly with a second composition comprising a second therapeutic or prophylactic agent for coronavirus infection. In some embodiments compositions of the present technology are administered concomitantly with a second vaccine against coronavirus infection, e.g., a second vaccine against the same coronavirus. In some such embodiments, the coronavirus is SARS-CoV-2.

As used herein, a “therapeutically effective amount” or “an effective amount” refers to an amount of a composition, vaccine, antigen, or antibody that is sufficient to prevent or treat coronavirus infection, to alleviate (e.g., mitigate, decrease, reduce) at least one of the symptoms associated with coronavirus infection, and/or to induce an immune response to a coronavirus, such that benefit to the subject is provided. The effective amount of a composition, vaccine, antigen, or antibody may be determined by one of ordinary skill in the art. Exemplary dosage amounts for an adult human include, without limitation, from about 0.1 to 500 mg/kg of body weight of antigen or antibody per day, which may be administered in a single dose or in the form of individual divided doses, such as from 1 to 5 times per day, or weekly, or bi-weekly.

In some embodiments, an effective amount of a composition comprising a VLP contains about 0.05 to about 1500 μg, about 10 to about 1000 μg, about 30 to about 500 μg, or about 40 to about 300 μg, or any integer between those values. For example, a VLP may be administered to a subject at a dose of about 0.1 μg to about 200 mg, e.g., from about 0.1 μg to about 5 μg, from about 5 μg to about 10 μg, from about 10 μg to about 25 μg, from about 25 μg to about 50 μg, from about 50 μg to about 100 μg, from about 100 μg to about 500 μg, from about 500 μg to about 1 mg, or from about 1 mg to about 2 mg, with optional boosters given at, for example, 1 week, 2 weeks, 3 weeks, 4 weeks, two months, three months, 6 months and/or a year later.

A composition, vaccine, antigen or antibody may also be administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid). For prophylactic purposes, the amount of peptide in each dose is selected as an amount which induces an immunoprotective response without significant adverse side effects in a typical vaccine. Following an initial vaccination, subjects may receive one or several booster immunisations adequately spaced.

It will be understood that the specific dose level and frequency of dosage for any particular subject may be varied and will depend upon a variety of factors including the activity of the specific compound employed, the metabolic stability and length of action of that compound, the species, age, body weight, genusl health, sex and diet of the subject, the mode and time of administration, rate of excretion and clearance, drug combinations, and severity of the particular condition.

In one embodiment, there is provided use of the compositions or vaccines or molecules as described herein for detecting the presence of a coronavirus, e.g., SARS-CoV-2, in a subject. In another embodiment, there is provided a method of detecting the presence of a coronavirus, e.g., SARS-CoV-2 in a subject comprising: obtaining a sample from the subject, and assaying the sample for the presence of antibodies that bind specifically to the compositions or vaccines or molecules as described herein, to determine the presence of the coronavirus S-protein in the subject, wherein the presence of antibodies specific for the S-protein indicates presence of coronavirus in the subject. In another embodiment, there is provided use of the compositions or vaccines or molecules as described herein for detecting the presence of neutralizing coronavirus antibodies in a subject, using the compositions or vaccines or molecules as described herein as a reagent in a cellular assay to detect neutralizing antibodies in a sample from the subject.

Also within this disclosure are kits comprising an effective amount of a compositions or vaccines or molecules as described herein. A kit can include one or more other elements including: instructions for use; other reagents, e.g., a detectable label, a therapeutic agent, etc; devices or other materials for preparing the compositions or vaccines or molecules as described herein for administration; pharmaceutically acceptable carriers; adjuvants; and devices or other materials for administration to a subject.

The container means of the kits will genuslly include at least one vial, test tube, flask, bottle, syringe or other container means, into which a component may be placed, and preferably, suitably aliquoted. Where there is more than one component in the kit, the kit also will genuslly contain a second, third or other additional container into which the additional components may be separately placed. However, various combinations of components may be comprised in a vial. A kit will also include instructions for employing the kit components as well the use of any other reagent not included in the kit. Instructions may include variations that can be implemented.

The technology described herein is not meant to be limited to particular methods, reagents, compounds, compositions or biological systems, which can, of course, vary. It should also be understood that terminology used herein is for the purpose of describing particular aspects only, and is not intended to be limiting.

The examples below are given to illustrate the practice of various embodiments of the present disclosure. They are not intended to limit or define the entire scope of this disclosure. It should be appreciated that the disclosure is not limited to the particular embodiments described and illustrated herein but includes all modifications and variations falling within the scope of the disclosure as defined in the appended embodiments.

### EXAMPLES

The examples below are given so as to illustrate the practice of various embodiments of the present disclosure. They are not intended to limit or define the entire scope of this disclosure. It should be appreciated that the disclosure is not limited to the particular embodiments described and illustrated herein but includes all modifications and variations falling within the scope of the disclosure as defined in the appended embodiments.

### Example 1: Design of E. Coli-Bacillus Bifunctional Cloning Vector without an Antibiotic-Resistance Gene

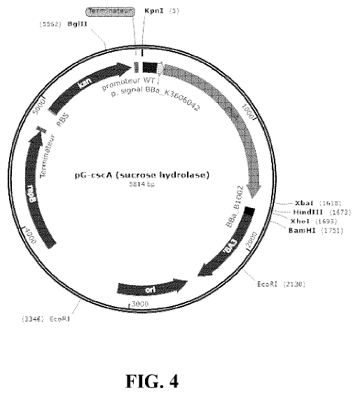

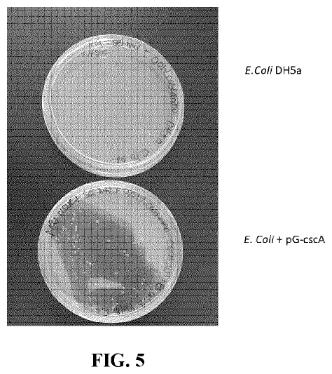

Cloning vector with selection on sucrose, in Escherichia coli. In order to eliminate the use of antibiotics in the selection of transformants for the step in E. coli, the coding gene for the sucrose hydrolase (cscA) as well as its natural promoter in Escherichia coli B-62 (NCBI accession number AF084030.1) are synthesized by adding the peptide signal NSP4 from the BioBrick system (BBa_K3606042), a transcription terminator (BBa_B1002). The expression of the gene is done under the control of the strong constitutive promoter, BBa_J23100, to which the RBS BBa_B0030 has been added upstream. The different constructs were synthesized by IDT (gBlocks® Gene Fragments, Integrated DNA Technologies Inc, USA). In order to test the potential of the selection system, Kpnl andXbal restriction sites were added to the ends of the synthesis gene in order to perform a directed cloning in a vector containing a resistance to kanamycin (FIG. 4). The vector containing the sucrose hydrolase is transformed in DH5a E. coli chemically competent cells by following a standard transformation protocol by thermal shock. The transformants are selected on Petri LB Broth (Lennox) with kanamycin 40 ug/ml. PCR colonies are then realized on the colonies by using primers specifically targeting the sucrose hydrolase gene. The positive clones are then transplanted in a M9 minimal medium with sucrose and in the absence of kanamycin (for 200 ml of distilled water, adding Na₂HPO₄: 1.2 g, KH₂PO₄: 0.6 g, NaCl: 0.1 g, NH₄Cl: 0.2, Agar: 3 g, MgSO₄ 1M: 200 μl, CaCl₂ 0.1 M: 200 μl, Sucrose 1% and Casamino acid: 0.01%) with the addition of 1× of a recipe of salt mix 1000× (for 1000 ml of distilled water, the addition of boric acid: 0.062 g, CuSO4: 0.05 g, MnSO₄: 0.127 g, Na2MoO4: 0.029 g and NH₄Cl: 1.07 g). The Petri dishes are incubated at 37° C. until obtaining colonies (FIG. 5). The wild DH5a E. coli strain is used as a control. The results obtained show that the wild DH5a strain, as well as the construct without peptide signal are incapable of growing on M.M+sucrose. However, clones that have the pG-cscA plasmid (with peptide signal) are able to grow on the same medium in less than 5 days.

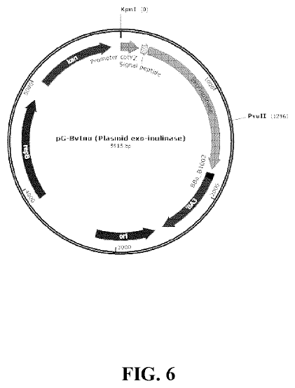

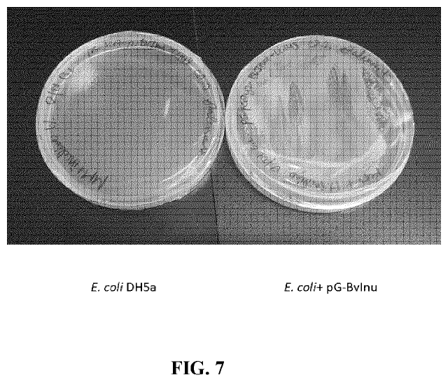

Cloning vector with selection on Inulin in Bacillus subtilis. In order to eliminate the use of antibiotics in the selection of transformants in B. subtilis, the coding gene for an exo-inulinase (Bvlnu) of Bacillus spp (NCBI accession number MK243491.1) was synthesized by adding the promoter cotYZ (Part:BBa_K2114000), the peptide signal of the inulinase gene originating from the B. velezensis U50 strain and a transcription terminator (BBa_B1002). The construct was synthesized by IDT (gBlocks® Gene Fragments, Integrated DNA Technologies Inc, USA). To test the selection system, a preliminary step of amplification by PCR reaction for the synthetic fragment of exo-inulinas Bvlnu is carried out with primers having an overhang of 25 base pairs with a vector containing a resistance to kanamycin. The vector is digested with the Xbal and Xhol restriction enzymes followed by an DNA assembly reaction (NEB DNA assembly kit) with the PCR fragment from the Bvlnu synthetic gene previously obtained (FIG. 6). The vector containing the exo-inulinase is transformed in the chemically competent E. coli DH5A cells following a protocol for transformation by thermal shock. The transformants are selected on Petri LB Broth (Lennox) with kanamycin 40 ug/ml. PCR colonies are then realized on colonies using primers that specifically target the Bv-inulinase gene. The positive clones in the PCR colony are then placed in a liquid culture in a 2×Ty plus kanamycin 40 ug/ml medium (Yeast extract: 1%, Peptone: 1.6% and NaCl: 0.5% and incubated at 37° C. for 16 hours. After incubation, a plasmid extraction is carried out and the vector is transformed in the Bacillus subtilis U148 strain of the Ulysse Biotechnologies collection. The transformation is carried out according to the following protocol: prepare a B. subtilis U148 culture in a rich, TB standard medium (37° C. for 18 hours at 240 rpm). Use the Bartels et al. (2018) transformation protocol [90]. Use 1 ug of plasmid DNA at the time of transformation. Selection is carried out on a rich+kanamycin 10 ug/ml medium. PCR colonies are then realized on colonies using primers specifically targeting the Bv-inulinase gene. The positive clones in the PCR colony are then plated in a M9 minimal medium with inulin and in the absence of kanamycin (for 200 ml of distilled water, addition of Na2HPO4: 1.2 g, KH2PO4: 0.6 g, NaCl: 0.1 g, NH4Cl: 0.2 g, Agar: 3 g, MgSO4 1M: 200 μl, Cacl2 0.1 M: 200 μl and Inulin 1%) with the addition of 1× a 1000× salt mix recipe (for 1000 ml of distilled water with the addition of boric acid: 0.062 g, CuSO4: 0.05 g, MnSO₄: 0.127 g, NaMoO4: 0.029 g and NH4Cl: 1.07 g). The Petri dishes are incubated at 37° C. until obtaining colonies (FIG. 7). A wild Bacillus subtilis strain is used for the control. The results obtained show that the wild B. subtilis U148 strain is incapable of growing in M.M.+inulin while the transformed strain is capable of doing so.

### Example 2: Bidirectional Cloning Vector in E. coli and Bacillus subtilis without Antibiotic-Resistance Genes

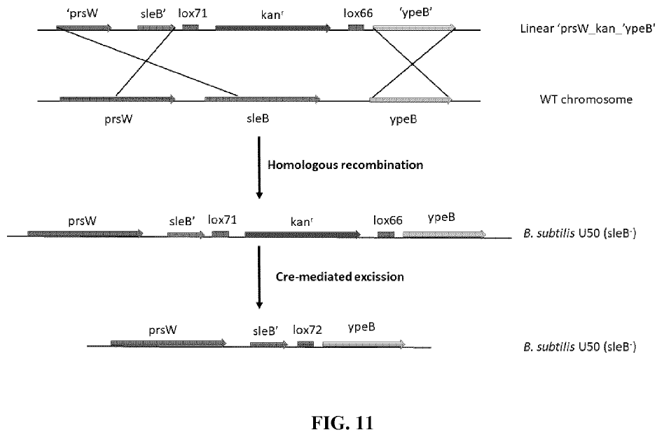

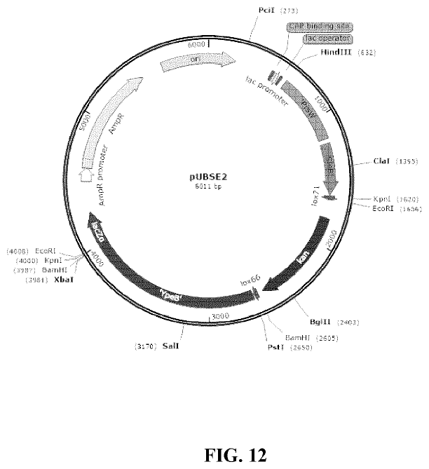

The synthetic sucrose hydrolase (cscA) as well as the exo-inulinase (Bvlnu) genes, described above, are combined together in the different vectors used for the spore display of different epitopes presented in this disclosure. The final objective is to be capable of realizing the selection of the different transformants in selection mediums without antibiotics, in both E. coli and B. subtilis (and its derivatives). A PCR reaction is carried out on the two synthesized genes as well as the on the “SARS-CoV-2” constructs by using primers having overhang in 25 base pairs, allowing the integration of the two genes in the different vectors between the repB gene and the origin of replication of E. coli by DNA assembly reaction (NEB DNA assembly kit). The different constructs now containing the sucrose hydrolase and the exo-inulinase are transformed in chemically competent E. coli DH5a cells following a protocol for transformation by thermal shock. The transformants are selected on Petri LB Broth (Lennox) with kanamycin 40 ug/ml. PCR colonies are then realized using primers specifically targeting the sucrose hydrolase as well as the Bv-inulinase. The positive clones are then plated in a 2×Ty standard medium+kanamycin 40 ug/ml (Yeast extract: 1%, Peptone: 1.6% and NaCl: 0.5%) and incubated at 37° C. for 16 hours. After plasmid extraction, a PCR is performed in order to remove the kanamycin-resistance gene. The PCR product is then purified and the vector is recircularized following a KLD reaction (KLD enzyme mix of NEB). (FIG. 11 and FIG. 12). The constructs without kanamycin-resistance genes are then transformed again in chemically competent E. coli DH5a cells following a protocol for transformation by thermal shock. The transformation is cleaned by two successive washings and using a 0.85% sterile saline solution. The pellet is again resuspended in the initial volume then spread onto a M9 minimal medium with sucrose 1% as the carbon source (see recipe above). The petri dishes are incubated at 37° C. until obtaining colonies. The PCR colonies are then realized on the colonies by using primers specifically targeting the coding gene for sucrose hydrolase cscA. The positive clones are then incubated in a standard 2×TY medium supplemented with 1% sucrose. The cultures are incubated at 37° C. for 16 hours to extract the plasmids. The different constructs are then transformed in B. subtilis U148. The different transformations are cleaned by two successive washings using a 0.85% sterile saline solution. The pellet is resuspended in the initial volume then spread onto a M9 minimal medium+inulin 1% (see recipe above). The petri dishes are then incubated at 37° C. until obtaining colonies. The transformants are selected in a M9 minimal medium+1% inulin. PCR colonies are then realized using primers specifically targeting the sucrose hydrolase cscA genes as well as the Bvlnu exo-inulinase genes. Positive clones are then stored at −80° C.

### Example 3: Plasmid Stability Assay

The segregational stability of the different clones is then evaluated in the following manner. The clones are plated on rich petri dishes and incubated for 18 hours at 37° C. in order to obtain isolated colonies. The next day, PCR colonies are realized in order to confirm the presence of sucrose hydrolase and Bvlnu exo-inulinase genes. Select a positive colony and transfer it in a sterile manner into a standard rich liquid medium (Terrific broth). Incubate at 37° C. for 18 hours at 240 rpm. The cultures are then diluted 1:10,000 in a fresh TB medium with sucrose and inulin. This process is repeated for 7 days. Each day, a count on the solid medium is performed in order to obtain isolated colonies. Ten PCR colonies using primers that make it possible to amplify cscA and Bvlnu genes are realized. The results obtained show that after four rounds of transplantation, 100% of the colonies tested remain positive. After 10 days, a level of instability between 40% and 60% is noted, according to the clones tested. This instability is probably due to the type of origin of replication used (Staphylococcus aureus repB). Indeed, it has been shown that repB normally functions in B. subtilis, but that a large number of plasmids based on the principle of the rolling circle related to this origin of replication do not hold up very long in transformed Bacillus strains. Zhao et al. (2020) demonstrated that the orientation as well as the positioning of the BA3 fragment (that corresponds to a single strand origin SSO normally present in the systems based on the rolling circle) have an important impact on the stability and the number of copies of the plasmid transformed in Bacillus [91]. In the present case, given that the stability is adequate over 4 successive transfer cycles, the impact is negligible, because the final objective is to produce “eternal” spores in one single fermentation cycle. Additionally, PCR colonies have been realized after fermentation and the results showed a plasmid stability of more than 95%.

### Example 4: Design of an E. coli-Bacillus Bifunctional Cloning Vector Expressing a Phospholipase on the Surface of Bacillus Spores as a Rapid Detection System on Solid Medium

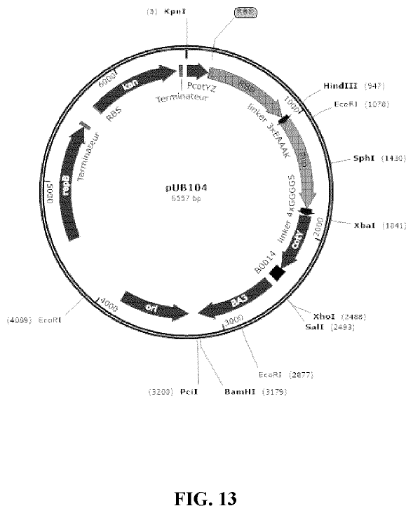

The DNA of the vector pG7k was used as a receptor plasmid for the synthetic elements containing the coding gene for Bacillus cereus phospholipase C activity to be cloned. Based on the work of Bartels et al. (2018), it was shown that PcotYZ is a strong promoter that is expressed in the middle of the growth exponential phase of Bacillus [90]. PcotYX is a constitutive promoter that has proven to be very effective in promoting the expression of coding genes for proteins in the cot family which, once produced, will position themselves in the last outer layer (crust) of the spores. The cotY protein has proven to be a good choice to fuse a protein of interest thereto so that the latter can be displayed on the surface of the spores. A DNA fragment containing the following elements was synthesized: PcotYZ promoter+RBS+Plip+rigid linker+cotY+B0014 transcription terminator (FIG. 12). Phospholipase C activity is present only in B. cereus, B. thuringiensis and B. anthracis. Strains like B. subtilis and its subspecies, B. amyloliquefaciens, B. velezensis, B. circulans and any other Bacillus are not recognized as being able to produce this phospholipase. Phospholipase activity is easily detectable on a solid medium to which an egg yolk emulsion is added. A phospholipase positive strain will have a clearing zone around it, while a negative strain will have no clearing zone. To confirm the efficacy of displaying phospholipase C activity on the surface of a Bacillus spore, the transformants obtained in the B. velezensis U50 and B. subtilis U148 strains were screened on a nutrient agar medium+5 μg/ml kanamycin+egg yolk emulsion. FIG. 13 shows an example of dilution of competent cells of B. velezensis U50 transformed with the plasmid pUB104. The presence of the clearing zone around all the transformants confirms two things: a) the notion of display on a Bacillus spore works; b) the use of B. cereus phospholipase C activity as a means of detecting display on a naturally phospholipase negative Bacillus spore works. The same approach was used with success for the B. subtilis U148 strain as well as with other Bacillus belonging to the Bacillus amyloliquefaciens, Bacillus licheniformis, Priesta megaterium (previously known as Bacillus megaterium) and Bacillus circulans (data not shown) among others.

### Example 5: Preparation of Bacillus “Eternal” Spore Using Homologous Recombination to Knockout B. subtilis U50 sleB Gene

To block Bacillus spore germination, a linear DNA sequence was constructed to the knockout B. subtilis U50 sleB gene. The same experiment was reproduced for the strain B. velezensis U148. FIG. 11 represents this linear DNA sequence and the homologous recombination occurring with B. subtilis U50's chromosome. Linear DNA sequence was constructed in the following manner. First, the lox71_kan_lox66 DNA sequence was synthesized and amplified by PCR. This sequence contains a kanamycin resistance gene flanked by lox71 on the 5′ side and lox66 on the 3′ side. Then, “prsW-sleB” and “ypeB” genes were amplified by PCR using WT B. subtilis U50 genomic DNA as a template. Those PCR fragments were added on each side of the lox71_kan_lox66 DNA sequence using Gibson assembly. PCR primers were designed to add a 25 bp overhang with adjacent DNA fragments to allow assembly. The resulting assembly was amplified by PCR to obtain the linear DNA sequence “prsW_kan ‘ypeB”. The linear DNA sequence “prsW_kan_‘ypeB” was inserted in pUC19, a commonly used cloning vector. The linear DNA sequence “prsW_kan_‘ypeB” and pUC19 were cut with HindIII and Xbal, and then linked, to produce pUBSE2 (FIG. 12). Resulting plasmid was amplified using DH5a competent E. coli, then linearized using ScaI restriction enzyme. Linearized pUSE2 plasmid was transformed in B. subtilis U50 to allow homologous recombination with the WT chromosome. Linearized pUSE2 plasmid is 6011 bp, which is an adequate length for genome editing of B. subtilis [92]. The kanamycin resistance gene allows selection of B. subtilis U50 with successful homologous recombination. Chromosomal integration is subsequently confirmed by PCR.

A Cre-mediated excision can then remove the kanamycin resistance gene from the B. subtilis U50 chromosome [93]. The pCRE plasmid was constructed to contain a Cre isopropyl-β-D-thiogalactopyranoside (IPTG)-inducible expression cassette, a replication origin for plasmid replication in E. coli (ORI) and B. subtilis (repB), an ampicillin resistance gene allowing selection of E. coli transformants and a chloramphenicol resistance gene allowing selection of B. subtilis transformants. The pCRE plasmid is then cured after a few subcultures on nutrient agar without antibiotics. The germination rate of the mutant U50 sleW-strain is determined by comparing with the U50 WT strain. The bacteria were fermented separately (Labfors 13 liters) in order to obtain a sporulation yield greater than 98%. A 50 ml sample of each strain is then concentrated and washed three times with sterile physiological saline. The pellets are resuspended in 50 ml of saline. The amount of bacteria is determined by conventional counting. An Erlenmeyer flask of rich medium is then inoculated to obtain an initial count of 1×10E5 cfu/ml. Cultures are incubated at 37° C. for 18 h. A sample of each culture is pasteurized at 65° C. for 30 min. Counting of pasteurized and unpasteurized samples is then carried out. After pasteurization, the U50 WT strain has a count of less than 100 cfu/ml while the unpasteurized sample has a count of more than 5×10E9 cfu/ml. The U50 sleW-mutant strain has a count of nearly 1×10E5 for both the pasteurized and unpasteurized samples. The germination rate would be less than 0.05%.

### Example 6: Cloning of the Receptor Binding Domain of SARS-CoV-2 with Plip (South Africa Variant)

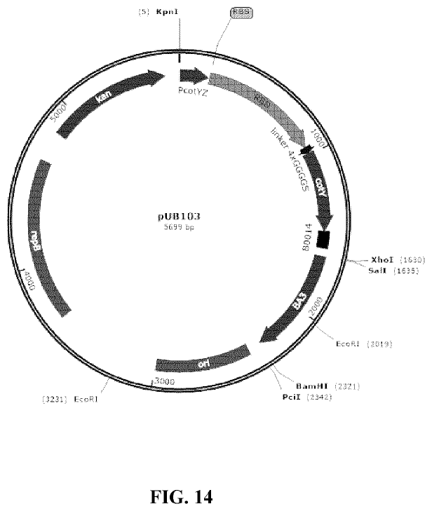

FIG. 13 shows the pUB104 plasmid that contains, in order: PcotYZ-RBD_{SF}-Plip-cotY. The nucleotide sequence of the receptor binding domain (RBD) was optimized to correspond to the codon-use table for B. subtilis. The amino acid sequence corresponding to the South Africa variant (N501Y and E484K). The synthetic fragments (Integrated DNA Technologies, Inc.) were then assembled with the pG7K vector at the PstI site that was not recreated after assembly using the NEB Gibson assembly kit. The two fragments have an overhang of 25 bases. The addition of a rigid linker was preferred between cotY and Plip in order to prevent the mature Plip protein from being trapped at the outer layer of the spore. The only function of the presence of the Plip phospholipase is to facilitate the rapid detection of the presence of protein fusion displayed on the surface of the Bacillus spores. When the phospholipase activity is confirmed (see FIG. 13 as an example) and the presence of the RBD is observed by flow cytometry and Western Blot, the coding gene for Plip is then eliminated by PCR to obtain the pUB103 construct (FIG. 14).

### Example 7: Fusion of the Multiepitope to the Receptor Binding Domain of pUB103

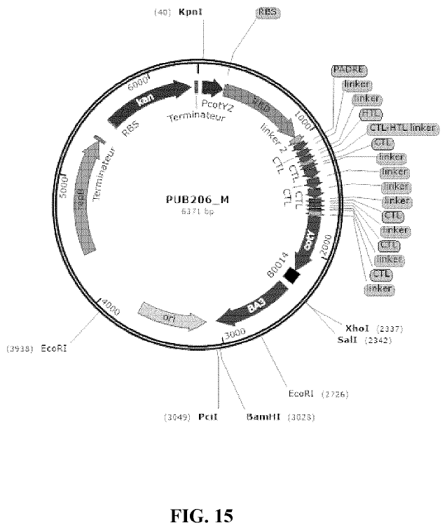

The optimized nucleotide sequence (B. subtilis codon Table) of the 8 epitopes selected and mentioned above was synthesized by Integrated DNA Technologies, Inc., foreseeing 25 overhang bases with the pUB103 clone that has been linearized by PCR. The Gibson assembly is carried out between the RBD and cotY of the pUB103 linearized by PCR. The final assembly makes it possible to obtain the following order: PcotYZ—RBD—multi-epitopes—cotY. The plasmid obtained is described in FIG. 15 (pUB206-M). The success of the assembly reaction of the two fragments is confirmed by PCR before transforming DH5α. The transformants are tested once again by PCR colony. The positive clones are then placed overnight in a liquid culture (2×TY+kan40) to isolate the plasmid. B. subtilis U148 and B. velezensis U50 are then transformed using the method described in Example 10.

### Example 8: Cloning of the Synthetic DNA of the PADRE-LTB Fragment in pUB103_deIRBD

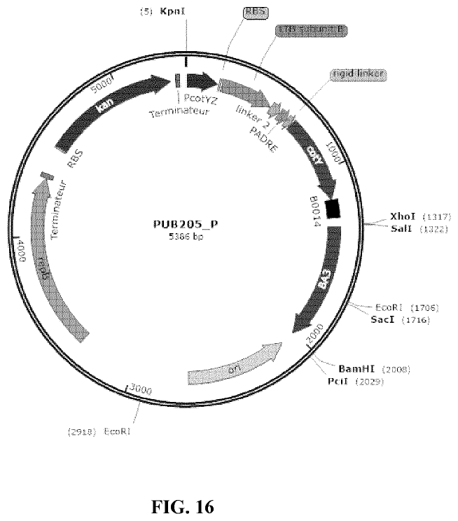

In the goal of amplifying immune response, it has been shown that the PADRE and LTB sequence expressions make it possible to obtain an effective adjuvant effect. It is with this in mind that the inventors have expressed the following combination of the outer surface of the Bacillus spores: cotY—PADRE—LTB. The synthetic PADRE—LTB fragment optimized to respect the Table of use of the Bacillus codons was cloned in the pUB103 plasmid that has been previously linearized by PCR in order to promote the assembly of 25 available overhang bases on each end of the synthetic gene. The plasmid obtained is described in FIG. 16 (pUB205-P). The success of the assembly reaction of the two fragments is confirmed by PCR before transforming DH5α. The transformants are tested once again by PCR colony. The positive clones are then placed overnight in a liquid culture (2×TY+kan40) to isolate the plasmid. B. subtilis U148 and B. velezensis U50 are then transformed using the method described in Example 10.

### Example 9: Fusion of the Multiepitope to the Omicron Receptor Binding Domain of pUB103

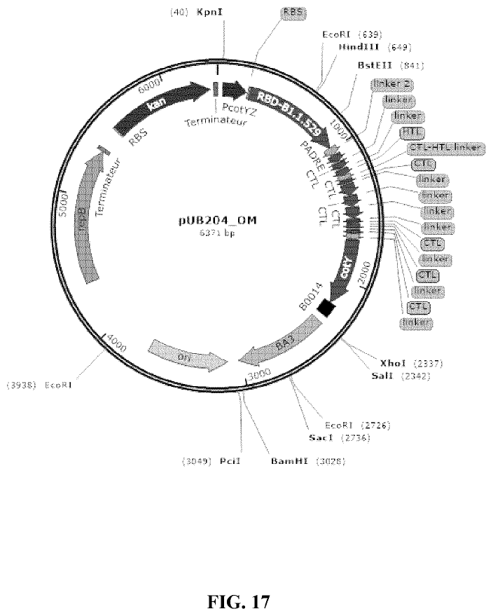

In order to clone the Omicron RBD synthetic gene, the inventors used pUB204-M plasmid in which the initial RBD has been eliminated by PCR and replaced by the Omicron RBD. The final construct obtained (pUB204-OM) is shown in FIG. 17. The success of the assembly reaction of the two fragments is confirmed by PCR before transforming DH5α. the transformants are tested once again by PCR colony. The positive clones are then placed overnight in a liquid culture (2×TY+kan40) to isolate the plasmid. B. subtilis U148 and B. velezensis U50 are then transformed using the method described in Example 10.

### Example 10: Transformation of Genus Bacillus

Starting from an isolated colony from a 24-hour fresh petri dish, inoculate 25 ml of TB (Terrific Broth) medium. Incubate 18 h at 37° C. while stirring at 220 rpm. The next morning, inoculate 10 ml of the MNGE medium with 0.2 ml of the overnight culture [90]. Incubate until an O.D.600 between 1.2 and 1.7 is obtained. Transfer 0.5 ml to a sterile 50 ml tube and add 1 μg of plasmid and 0.1 ml of expression mix (500 μl of 5% yeast extract; 250 μl of 10% casamino acid; 50 μl 5 mg/ml tryptophan; 250 μl water). Incubate at 220 rpm at 37° C. for 60 min. Plate 100 ul, 150 and 250 ul on PCA agar+5 ug/ml kanamycin. Incubate overnight at 37° C.

### Example 11: Production of Spores of Genus Bacillus in Fermenters

The various clones expressing on the surface of Bacillus subtilis U148 spores (or any other bacillus strains) were fermented for 72 hours in a rich medium (ratio C:N of 1,5:1) supplemented with trace mineral salts. The salt solution used (sodium molybdate, manganese sulfate, copper sulfate and boric acid) is added after sterilization of the culture medium. The fermenters are inoculated with a pre-culture incubated at 37° C. for 18 h (1 part of the culture: 100 parts of the culture medium to be inoculated). The dissolved oxygen concentration is kept constant at 20% by varying the stirring speed. The pH of the culture medium is not controlled during the first 6 hours of growth. Subsequently, the pH is maintained at 6.9. Growth and sporulation rates are assessed daily. The fermentation is stopped after 72 hours when the sporulation rate has reached more than 95%.

### Example 12: Expression of a Fluorescent Protein (Green Fluorescent Protein; GFP) on the Surface of Bacillus Spores

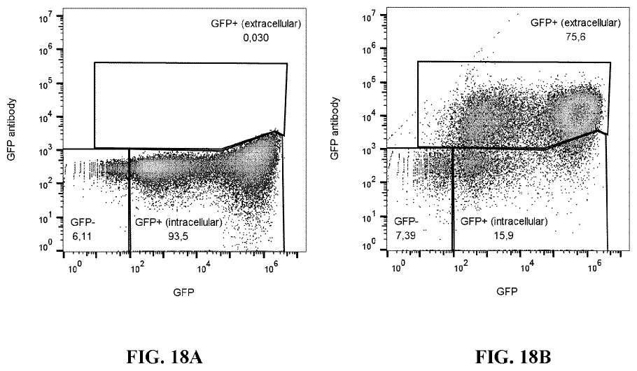

As a second proof of concept of the display system, the GFP was used. The inventor chose this protein because by comparing the fluorescence attributable du GFP and the one induced by the binding of an antibody, it was possible to assess the number of cells expressing the GFP intracellularly vs. extracellularly. Unfortunately, commercial specific antibodies for the antigens used in the conception of vaccines are often unavailable of ineffective, so it was important to test the robustness of the expression system. For this demonstration, 1×10⁶ GFP expressing vegetative Bacillus U50 cells were subjected to antibody labeling. The primary and secondary antibodies were diluted in PBS+0,5% BSA buffer (FACS buffer). The cells were incubated for 30 minutes with 50 uL if diluted primary antibody or only FACS buffer at room temperature, washed twice with PBS, then both samples were incubated 30 minutes in the dark with a secondary antibody. The cells were washed again twice, resuspended in PBS and analyzed on a flow cytometer (Accuri C6 Plus, Becton Dickinson Co. USA). As shown in FIGS. 18A and 18B, the GFP signal was detectable in more than 90% of U50-GFP cells, with over 82% expressing it extracellularly.

### Example 13: Expression of E. coli Heat-Labile Enterotoxin B Chain (LTB) on the Surface of Spores

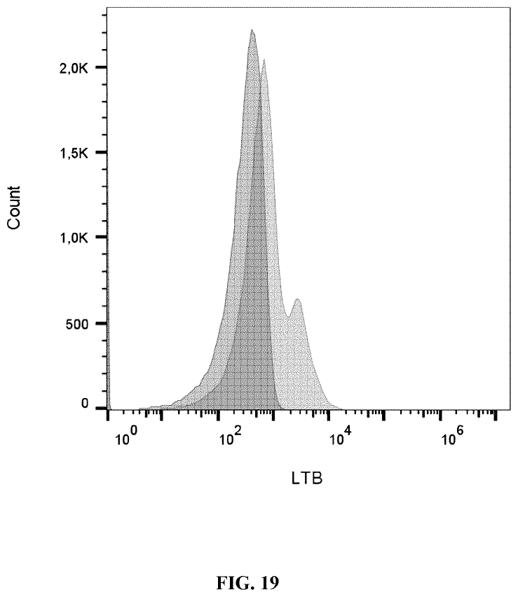

To be effective in a vaccine, the adjuvant must be exposed. To prove this fact, a flow cytometry assay was designed to detect the present of LTB at the surface of transformed U148 spores. Briefly, the spores were washed in PBS twice, and 1×10⁶ spores were subjected to antibody labeling. The primary antibodies were diluted to a concentration of 10 ng/μL in PBS+0,5% BSA buffer (FACS buffer) and the secondary diluted a thousand times in FACS buffer. One sample was incubated with a rabbit IgG isotype control, and another was marked with a polyclonal rabbit antibody raised against LTB. The cells were incubated for 30 minutes with 50 uL if diluted primary antibody at room temperature, washed twice with PBS, then incubated 30 minutes in the dark with a secondary antibody. The cells were washed again twice, resuspended in PBS and analyzed on a flow cytometer (Accuri C6 Plus, Becton Dickinson Co. USA). As shown in FIG. 19, the LTB was detected on the surface in a least a third of Bacillus U148 spores transformed with pUB205-P plasmid.

### Example 14: Expression of the Receptor Binding Domain (RBD) of SARS-CoV-2 on the Surface of Spores

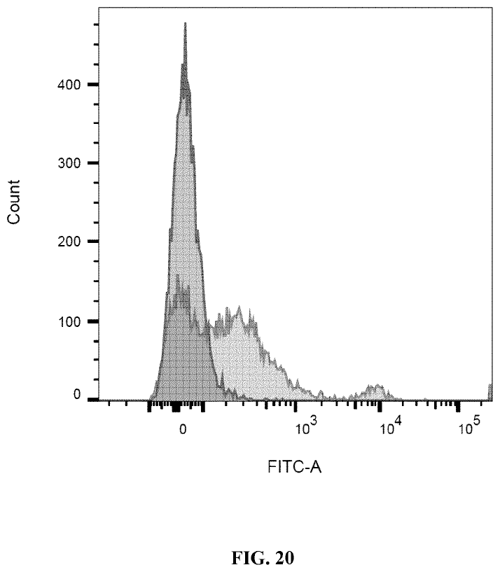

To adequately trigger a humoral immune response, and potentially lead to the production of neutralizing antibodies, an antigen is to be displayed. To prove the fact that the RBD is indeed exposed at the surface of Bacillus spores, a flow cytometry assay to compare the binding of specific RBD antibodies on the surface of transformed and wild-type U50. Briefly, the spores were washed in PBS twice, and 1×10⁶ spores were subjected to antibody labeling. The primary and secondary antibodies were diluted to a concentration of FACS buffer. The cells were incubated for 30 minutes with diluted primary antibody on ice with the primary antibody, washed with FACS buffer, then incubated 30 minutes on ice in the dark with a secondary antibody. The cells were washed again, resuspended in FACS buffer and analyzed on a flow cytometer (Fortessa X-20, Becton Dickinson Co. USA). As shown in FIG. 20, the RBD was detected at the surface of more than 50% of Bacillus U50 spores transformed with pUB104 plasmid.

### Example 15: Mice Immunization Test

6-8 week old female Balb/c mice were divided into 5 groups of 9 mice after a 5-day acclimatization period. Pre-immune serum samples were taken from each mouse before immunization. The animals then received the treatments as indicated in Table 2:

TABLE 2
Details of the different treatments in the in vivo assay

| | Adminis- | | | | Number of |
|---|---|---|---|---|---|
| Group | tration | Dose | Route | Regimen | animals |
| Oral-CTRL | UB50 | 1E9 in | Force- | 1×/day for | 9 |
| | | 200 uL | feeding | 3 days on | |
| Oral-Spicule | UBBV01 | 1E9 in | | days: 1-2-3 | 9 |
| | | 200 uL | | and 16 17-18 | |

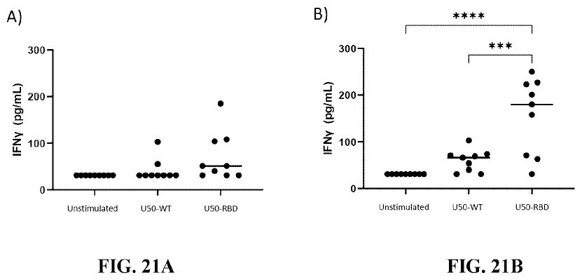

The isolation of the animals' splenocytes made it possible to detect a specific response to the antigen contained in the spores (FIG. 21). This was able to be determined by interferon-gamma assay (IFNγ) in the culture medium following the in vitro challenge of the isolated splenocytes of mice immunized with wild U50 (U50-WT; FIG. 21A) and U50 expressing the RBD (U50-RBD; FIG. 21B). IFNγ is a pre-inflammatory cytokine implicated notably in the defense against viral infections. As represented in FIG. 21A, the challenge of the splenocytes from mice immunized with U50-WT, whether it was with U50-WT or U50-RBD, did not induce a significant increase in the production of IFNγ. However, a significant increase in the production of cytokine was noted when the U50-RBD spores were used for the in vitro challenge of splenocytes from mice immunized with U50-RBD, which was not observed during the challenge of the splenocytes of this group of mice with the U50-WT spores.

All references cited in this specification, and their references, are incorporated by reference herein in their entirety where appropriate for teachings of additional or alternative details, features, and/or technical background.

While the disclosure has been particularly shown and described with reference to particular embodiments, it will be appreciated that variations of the above-disclosed and other features and functions, or alternatives thereof, may be desirably combined into many other different systems or applications. Also, that various presently unforeseen or unanticipated alternatives, modifications, variations or improvements therein may be subsequently made by those skilled in the art which are also intended to be encompassed by the following claims.

### BIBLIOGRAPHY

1. Williams, T. C. and W. A. Burgers, SARS-CoV-2 evolution and vaccines: cause for concern?Lancet Respir Med, 2021. 9(4): p. 333-335.
2. Rostami, A., et al., Display of B. pumilus chitinase on the surface of B. subtilis spore as a potential biopesticide. Pestic Biochem Physiol, 2017. 140: p. 17-23.
3. Nguyen, T. P. A., et al., Application ofyeastsurface display system in expression ofrecombinant pediocin PA-1 in Saccharomyces cerevisiae. Folia Microbiol (Praha), 2020. 65(6): p. 955-961.
4. Bilich, T., et al., T cell and antibody kinetics delineate SARS-CoV-2 peptides mediating long-term immune responses in COVID-19 convalescent individuals. Sci Transl Med, 2021. 13(590).
5. Dimitrov, I., et al., AllergenFP: allergenicity prediction by descriptor fingerprints. Bioinformatics, 2014. 30(6): p. 846-51.
6. Stamatopoulou, M., et al., Rapid decay of anti-SARS-CoV-2 antibodies in infection-naïve healthcare workers four months after vaccination. Germs, 2021. 11(4): p. 625-629.
7. Dutta, N. K., K. Mazumdar, and J. T. Gordy, The Nucleocapsid Protein of SARS-CoV-2: a Target for Vaccine Development. J Virol, 2020. 94(13).
8. Li, M. Y., et al., Expression of the SARS-CoV-2 cell receptor gene ACE2 in a wide variety of human tissues. Infect Dis Poverty, 2020. 9(1): p. 45.
9. Yoshimoto, F. K., The Proteins of Severe Acute Respiratory Syndrome Coronavirus-2 (SARS CoV-2 or n-COV19), the Cause of COVID-19. Protein J, 2020. 39(3): p. 198-216.
10. Sette, A. and S. Crotty, Adaptive immunity to SARS-CoV-2 and COVID-19. Cell, 2021. 184(4): p. 861-880.
11. Nelde, A., et al., SARS-CoV-2-derived peptides define heterologous and COVID-19-induced T cell recognition. Nat Immunol, 2021. 22(1): p. 74-85.
12. Smith, C. C., et al., Landscape and selection of vaccine epitopes in SARS-CoV-2. Genome Med, 2021. 13(1): p. 101.
13. Rahmani, A., et al., Applying high throughput and comprehensive immunoinformatics approaches to design a trivalent subunit vaccine for induction of immune response against emerging human coronaviruses SARS-CoV, MERS-CoV and SARS-CoV-2. J Biomol Struct Dyn, 2021: p. 1-17.
14. Dimitrov, I., et al., AllerTOP v.2—a server for in silico prediction of allergens. J Mol Model, 2014. 20(6): p. 2278.
15. Gupta, S., et al., In silico approach for predicting toxicity of peptides and proteins. PLoS One, 2013. 8(9): p. e73957.
16. Rosa, D. S., et al., The pan HLA DR-binding epitope improves adjuvant-assisted immunization with a recombinant protein containing a malaria vaccine candidate. Immunol Lett, 2004. 92(3): p. 259-68.
17. Shrwani, K., et al., Detection of Serum Cross-Reactive Antibodies and Memory Response to SARS-CoV-2 in Prepandemic and Post-COVID-19 Convalescent Samples. J Infect Dis, 2021. 224(8): p. 1305-1315.
18. Velders, M. P., et al., Defined flanking spacers and enhanced proteolysis is essential for eradication of established tumors by an epitope string DNA vaccine. J Immunol, 2001. 166(9): p. 5366-73.
19. Yadav, S., et al., Design of a multi-epitope subunit vaccine for immune-protection against Leishmania parasite. Pathog Glob Health, 2020. 114(8): p. 471-481.
20. Tarrahimofrad, H., et al., Designing a multi-epitope vaccine to provoke the robust immune response against influenza A H7N9. Sci Rep, 2021. 11(1): p. 24485.
21. Pashine, A., N. M. Valiante, and J. B. Ulmer, Targeting the innate immune response with improved vaccine adjuvants. Nat Med, 2005. 11(4 Suppl): p. S63-8.
22. O'Hagan, D. T. and N. M. Valiante, Recent advances in the discovery and delivery of vaccine adjuvants. Nat Rev Drug Discov, 2003. 2(9): p. 727-35.
23. da Hora, V. P., et al., Non-toxic derivatives of LT as potent adjuvants. Vaccine, 2011. 29(8): p. 1538-44.
24. Simmons, C. P., et al., Immunomodulation using bacterial enterotoxins. Scand J Immunol, 2001. 53(3): p. 218-26.
25. Ma, Y., Recent advances in nontoxic Escherichia coli heat-labile toxin and its derivative adjuvants. Expert Rev Vaccines, 2016. 15(11): p. 1361-1371.
26. Hajishengallis, G. and T. D. Connell, Type II heat-labile enterotoxins: structure, function, and immunomodulatory properties. Vet Immunol Immunopathol, 2013. 152(1-2): p. 68-77.
27. Tacket, C. O., et al., Immunogenicity of recombinant LT-B delivered orally to humans in transgenic corn. Vaccine, 2004. 22(31-32): p. 4385-9.
28. Weltzin, R., et al., Parenteral adjuvant activities of Escherichia coli heat-labile toxin and its B subunit for immunization of mice against gastric Helicobacter pylori infection. Infect Immun, 2000. 68(5): p. 2775-82.
29. Ahren, C., M. Jertborn, and A. M. Svennerholm, Intestinal immune responses to an inactivated oral enterotoxigenic Escherichia coli vaccine and associated immunoglobulin A responses in blood. Infect Immun, 1998. 66(7): p. 3311-6.
30. Kaushik, H., et al., Immunization with recombinant fusion of LTB and linear epitope (40-62) of epsilon toxin elicits protective immune response against the epsilon toxin of Clostridium perfringens type D. AMB Express, 2019. 9(1): p. 105.
31. Tamura, S., et al., Effects of frequent intranasal administration of adjuvant-combined influenza vaccine on the protection against virus infection. Vaccine, 1997. 15(16): p. 1784-90.
32. Waheed, M. T., et al., Plastid expression of a double-pentameric vaccine candidate containing human papillomavirus-16 L1 antigen fused with LTB as adjuvant: transplastomic plants show pleiotropic phenotypes. Plant Biotechnol J, 2011. 9(6): p. 651-60.
33. Pitcovski, J., et al., Oral subunit SARS-CoV-2 vaccine induces systemic neutralizing IgG, IgA and cellular immune responses and can boost neutralizing antibody responses primed by an injected vaccine. Vaccine, 2022.
34. Liu, L., et al., Quantitative Proteomic Analysis of Escherichia coli Heat-Labile Toxin B Subunit (LTB) with Enterovirus 71 (EV71) Subunit VP1. Int J Mol Sci, 2016. 17(9).
35. Lee, C. H., G. Hajishengallis, and T. D. Connell, Dendritic Cell-Mediated Mechanisms Triggered by LT-IIa-B5, a Mucosal Adjuvant Derived from a Type II Heat-Labile Enterotoxin of Escherichia coli. J Microbiol Biotechnol, 2017. 27(4): p. 709-717.
36. Sung, J. C., et al., Expression of SARS-CoV-2 Spike Protein Receptor Binding Domain on Recombinant B. subtilis on Spore Surface: A Potential COVID-19 Oral Vaccine Candidate. Vaccines (Basel), 2021. 10(1).
37. Little, M., et al., Bacterial surface presentation of proteins and peptides: an alternative to phage technology? Trends Biotechnol, 1993. 11(1): p. 3-5.
38. Georgiou, G., et al., Practical applications of engineering gram-negative bacterial cell surfaces. Trends Biotechnol, 1993. 11(1): p. 6-10.
39. Gunneriusson, E., et al., Surface display of a functional single-chain Fv antibody on staphylococci. J Bacteriol, 1996. 178(5): p. 1341-6.
40. Samuelson, P., et al., Cell surface display of recombinantproteins on Staphylococcus carnosus. J Bacteriol, 1995. 177(6): p. 1470-6.
41. Schneewind, O., A. Fowler, and K. F. Faull, Structure of the cell wall anchor of surface proteins in Staphylococcus aureus. Science, 1995. 268(5207): p. 103-6.
42. Schreuder, M. P., et al., Targeting of a heterologous protein to the cell wall of Saccharomyces cerevisiae. Yeast, 1993. 9(4): p. 399-409.
43. Kang, M., et al., Display of quintuple glucagon-like peptide 1 (28-36) nonapeptide on Bacillus subtilis spore for oral administration in the treatment of type 2 diabetes. J Appl Microbiol, 2021. 130(2): p. 314-324.
44. Oh, Y., et al., Bacillus subtilis spore vaccines displaying protective antigen induce functional antibodies and protective potency. BMC Vet Res, 2020. 16(1): p. 259.
45. Kang, S. J., et al., Surface display of p75, a Lactobacillus rhamnosus GG derived protein, on Bacillus subtilis spores and its antibacterial activity against Listeria monocytogenes. AMB Express, 2020. 10(1): p. 139.
46. Li, W., et al., Surface Display of Antigen Protein VP8* of Porcine Rotavirus on Bacillus Subtilis Spores Using CotB as a Fusion Partner. Molecules, 2019. 24(20).
47. Cen, Q., et al., Immune evaluation of a Saccharomyces cerevisiae-based oral vaccine against Helicobacter pylori in mice. Helicobacter, 2021. 26(1): p. e12772.
48. Lei, H., et al., Haemagglutinin displayed on the surface of Lactococcus lactis confers broad cross-clade protection against different H5N1 viruses in chickens. Microb Cell Fact, 2020. 19(1): p. 193.
49. Isticato, R., E. Ricca, and L. Baccigalupi, Spore Adsorption as a Nonrecombinant Display System for Enzymes and Antigens. J Vis Exp, 2019(145).
50. Cho, E. A., E. J. Kim, and J. G. Pan, Adsorption immobilization of Escherichia coli phytase on probiotic Bacillus polyfermenticus spores. Enzyme Microb Technol, 2011. 49(1): p. 66-71.
51. Huang, J. M., et al., Mucosal delivery of antigens using adsorption to bacterial spores. Vaccine, 2010. 28(4): p. 1021-30.
52. Isticato, R., et al., Surface display of recombinant proteins on Bacillus subtilis spores. J Bacteriol, 2001. 183(21): p. 6294-301.
53. Ricca, E. and S. M. Cutting, Emerging Applications of Bacterial Spores in Nanobiotechnology. J Nanobiotechnology, 2003. 1(1): p. 6.
54. Wang, H., Y. Wang, and R. Yang, Recent progress in Bacillus subtilis spore-surface display: concept, progress, and future. Appl Microbiol Biotechnol, 2017. 101(3): p. 933-949.
55. Guoyan, Z., et al., Bacillus subtilis Spore Surface Display Technology: A Review of Its Development and Applications. J Microbiol Biotechnol, 2019. 29(2): p. 179-190.
56. Lin, P., et al., Progress in research and application development of surface display technology using Bacillus subtilis spores. Appl Microbiol Biotechnol, 2020. 104(6): p. 2319-2331.
57. Hsieh, H. Y., et al., A Bacillus Spore-Based Display System for Bioremediation of Atrazine. Appl Environ Microbiol, 2020. 86(18).
58. Kim, J.-h. S., KR), Kim, Byung-gee (Seoul, KR), Choi, Soo-keun (Daejon, KR), Jung, Heung-chae (Daejon, KR), Pan, Jae-gu (Daejon, KR), Method for expression of proteins on spore surface. 2009, Geneofocus Co., Ltd. (Daejon, KR): United States.
59. Kim, J.-h. S., KR), Kim, Byung-gee (Seoul, KR), Choi, Soo-keun (Daejon-city, KR), Jung, Heung-chae (Daejon-city, KR), Pan, Jae-gu (Daejon-city, KR), Methodfor expression of proteins on spore surface. 2004, KIM JUNE-HYUNG, KIM BYUNG-GEE, CHOI SOO-KEUN, JUNG HEUNG-CHAE, PAN JAE-GU: United States.
60. Perkins John B (CH), P. Z. C., Schyns Ghislain (CH), Spore Surface Display of Bioactive Molecules. 2013, DSM IP ASSETS BV (NL).
61. Acheson, D. W. K. N., MA), Sonenshein, Abraham L. (Brookline, MA), Keusch, Gerald T. (Lexington, MA), Bacterial spores as a heat stable vaccine delivery system. 1998, New England Medical Center Hospitals, Inc. (Boston, MA), Trustees, Of Tufts College (Medford, MA): United States.
62. Flick-smith, H. C. S., GB), Eyles, James Edward (Salisbury, GB), Waters, Emma Louise (Salisbury, GB), Walker, Nicola Jane (Salisbury, GB), Williamson, Ethel Diane (Salisbury, GB), Baillie, Leslie William (Salisbury, GB), Miller, Julie (Salisbury, GB), Vaccine formulation. 2012, The Secretary of State for Defence (GB): United States.
63. Thompson, B. C. C., MO, US), Siegel, Ashley (St. Louis, MO, US), Fusion proteins, recombinant bacteria, and methods for using recombinant bacteria. 2017, Spogen Biotech Inc. (St. Louis, MO, US): United States.
64. Goldman, S. W. C., CA, US), Lathrop, Stephanie J. (Sunnyvale, CA, US), Longchamp, Pascal F. (East Palo Alto, CA, US), Whalen, Robert G. (Foster City, CA, US), Methods and compositions for developing spore display systems for medicinal and industrial applications. 2002, Maxygen, Inc. (Redwood City, CA): United States.
65. Goldman, S. W. C., CA, US), Lathrop, Stephanie J. (Sunnyvale, CA, US), Longchamp, Pascal F. (East Palo Alto, CA, US), Whalen, Robert G. (Foster City, CA, US), Methods and compositions for developing spore display systems for medicinal and industrial applications. 2003, Maxygen Incorporated: United States.
66. Bhatt, R. B., CA, US), Horowitz, Lawrence (Atherton, CA, US), Estelles, Angeles (Belmont, CA, US), Spore associated display. 2009: United States.
67. Herrmann, J. E. N., MA, US), Belitsky, Boris R. (Swampscott, MA, US), Sonenshein, Abraham L. (Brookline, MA, US), Tzipori, Saul (Shrewsbury, MA, US), Methods, compositions and kits for vegetative cell-based vaccines and spore-based vaccines. 2011, TUFTS UNIVERSITY (Boston, MA, US): United States.
68. Liu, X. Z., et al., Enhancing expression of SSU1 genes in Saccharomyces uvarum leads to an increase in sulfite tolerance and a transcriptome profile change. FEMS Yeast Res, 2017. 17(3).
69. Sahin-Toth, M., Z. Lengyel, and H. Tsunekawa, Cloning, sequencing, and expression of cscA invertase from Escherichia coli B-62. Can J Microbiol, 1999. 45(5): p. 418-22.
70. Champney, W. S. and R. A. Jensen, D-Tyrosine as a metabolic inhibitor of Bacillus subtilis. J Bacteriol, 1969. 98(1): p. 205-14.
71. Geraskina, N. V., et al., The dtd gene from Bacillus amyloliquefaciens encodes a putative D-tyrosyl-tRNATyr deacylase and is a selectable marker for Bacillus subtilis. Microbiol Res, 2015. 171: p. 90-6.
72. Chen, X., J. L. Zaro, and W. C. Shen, Fusion protein linkers: property, design and functionality. Adv Drug Deliv Rev, 2013. 65(10): p. 1357-69.
73. Nezafat, N., et al., A novel multi-epitope peptide vaccine against cancer: an in silico approach. J Theor Biol, 2014. 349: p. 121-34.
74. Dolenc, I., E. Seemuller, and W. Baumeister, Decelerated degradation ofshortpeptides by the 20S proteasome. FEBS Lett, 1998. 434(3): p. 357-61.
75. Livingston, B., et al., A rational strategy to design multiepitope immunogens based on multiple Th lymphocyte epitopes. J Immunol, 2002. 168(11): p. 5499-506.
76. Nezafat, N., et al., Designing an efficient multi-epitope peptide vaccine against Vibrio cholerae via combined immunoinformatics and protein interaction based approaches. Comput Biol Chem, 2016. 62: p. 82-95.
77. Nain, Z., et al., Structural basis and designing of peptide vaccine using PE-PGRS family protein of Mycobacterium ulcerans—An integrated vaccinomics approach. Mol Immunol, 2020. 120: p. 146-163.
78. Ali, M., et al., Exploring dengue genome to construct a multi-epitope based subunit vaccine by utilizing immunoinformatics approach to battle against dengue infection. Sci Rep, 2017. 7(1): p. 9232.
79. Muhammad, S. A., et al., Polyvalent therapeutic vaccine for type 2 diabetes mellitus: Immunoinformatics approach to study co-stimulation of cytokines and GLUT1 receptors. BMC Mol Cell Biol, 2020. 21(1): p. 56.
80. Bergmann, C. C., et al., Flanking residues alter antigenicity and immunogenicity of multi-unit CTL epitopes. J Immunol, 1996. 157(8): p. 3242-9.
81. Wang, Q. M., et al., Epitope DNA vaccines against tuberculosis: spacers and ubiquitin modulates cellular immune responses elicited by epitope DNA vaccine. Scand J Immunol, 2004. 60(3): p. 219-25.
82. Bashkirov, V. I., N. V. Mil'shina, and A. A. Prozorov, [Nucleotide sequence and physical map of kanamycin-resistant plasmid pUB110 from Staphylococcus aureus]. Genetika, 1986. 22(7): p. 1081-92.
83. McKenzie, T., et al., The nucleotide sequence of pUB110: some salient features in relation to replication and its regulation. Plasmid, 1986. 15(2): p. 93-103.
84. Muller, R. E., et al., Complete nucleotide sequences of Bacillus plasmids pUB110dB, pRBH1 and its copy mutants. Mol Gen Genet, 1986. 202(1): p. 169-71.
85. Bernhard, K., H. Schrempf, and W. Goebel, Bacteriocin and antibiotic resistance plasmids in Bacillus cereus and Bacillus subtilis. J Bacteriol, 1978. 133(2): p. 897-903.
86. Setlow, B., E. Melly, and P. Setlow, Properties of spores of Bacillus subtilis blocked at an intermediate stage in spore germination. J Bacteriol, 2001. 183(16): p. 4894-9.
87. Ishikawa, S., et al., Regulation of a new cell wall hydrolase gene, cwlF, which affects cell separation in Bacillus subtilis. J Bacteriol, 1998. 180(9): p. 2549-55.
88. Sayer, C. V. and D. L. Popham, YpeB dimerization may be required to stabilize SleB for effective germination of Bacillus anthracis spores. BMC Microbiol, 2019. 19(1): p. 169.
89. Sun, R., et al., Germination-Arrest Bacillus subtilis Spores as An Oral Delivery Vehicle of Grass Carp Reovirus (GCRV) Vp7 Antigen Augment Protective Immunity in Grass Carp (Ctenopharyngodon idella). Genes (Basel), 2020. 11(11).
90. Bartels, J., et al., Sporobeads: The Utilization of the Bacillus subtilis Endospore Crust as a Protein Display Platform. ACS Synth Biol, 2018. 7(2): p. 452-461.
91. Zhao, X., et al., High copy number and highly stable Escherichia coli-Bacillus subtilis shuttle plasmids based on pWB980. Microb Cell Fact, 2020. 19(1): p. 25.
92. Wu, G., E. Drufva, and K. Wu, Fast genome editing in Bacillus subtilis. Eng Life Sci, 2019. 19(6): p. 471-477.
93. Yan, X., et al., Cre/lox system and PCR-based genome engineering in Bacillus subtilis. Appl Environ Microbiol, 2008. 74(17): p. 5556-62.

1. A recombinant microorganism comprising an antigen of SARS-CoV-2 expressed on the surface of the microorganism, wherein the recombinant microorganism is capable of inducing an immune response against a SARS-CoV-2 infection in a subject.

2. The recombinant microorganism of claim 1, wherein the microorganism is bacterial.

3. The recombinant microorganism of claim 1 or 2, wherein the microorganism is a member of a Bacillus genus.

4. The recombinant microorganism of claim 3, wherein the microorganism is a spore from a member of a Bacillus genus.

5. The recombinant microorganism of claim 4, wherein the Bacillus is selected from Bacillus subtilis, Bacillus circulans, Bacillus clausii, Bacillus amyloliquefaciens and Bacillus velezensis.

6. The recombinant microorganism of claim 5, wherein the Bacillus is Bacillus subtilis.

7. The recombinant microorganism of any one of claims 1 to 6, wherein the antigen of SARS-CoV-2 is selected from i) the S protein of SARS-CoV-2 or a portion thereof, ii) the N protein SARS-CoV-2 or a portion thereof, iii) the M protein of SARS-CoV-2 or a portion thereof, iv) the E protein of SARS-CoV-2 or a portion thereof, and iii) a combination or fusion of any one of i), ii), iii), and iv).

8. The recombinant microorganism of claim 6, wherein the portion thereof includes a portion of a receptor binding domain of the S protein, the N protein, the M protein, or the E protein.

9. The recombinant microorganism of any one of claims 1 to 6, wherein the antigen of SARS-CoV-2 is selected from: i) the Nsp-16 protein of SARS-CoV-2 or a portion thereof, ii) the Orf3a protein SARS-CoV-2 or a portion thereof, iii) the Orf3b protein of SARS-CoV-2 or a portion thereof, iv) the Orf6 protein of SARS-CoV-2 or a portion thereof, v) the Orf7a protein of SARS-CoV-2 or a portion thereof, vi) the Orf7b protein of SARS-CoV-2 or a portion thereof, vii) the Orf8 protein of SARS-CoV-2 or a portion thereof, viii) the Orf9b protein of SARS-CoV-2 or a portion thereof, iv) the Orf10 protein of SARS-CoV-2 or a portion thereof, and x) a combination or fusion of any one of i)-x).

10. The recombinant microorganism of any one of claims 1 to 6, wherein the antigen of SARS-CoV-2 comprises one or more of the amino acid sequences:
i)
AGLFQRHGEGTKATVGEPV;
ii)
KDGIIWVATEGALNT;
iii)
LSYYKLGASQRVAGD;
iv)
QYIKWPWYI;
v)
LLQFAYANRNRFLYIIKLIFLWLLWPV;
vi)
FIASFRLFARTRSMWSFNPETNILLN,
vii)
NSASFSTFKCYGVSPTKLNDLCFTNV;
viii)
TTDPSFLGRY;
and
ix)
KTFPPTEPKKAAYALSKGVHFV.

11. The recombinant microorganism of any one of claims 1 to 6, wherein the antigen of SARS-CoV-2 includes a fusion of epitopes related to different SARS-CoV-2 proteins.

12. The recombinant microorganism of any one of claims 1 to 6, wherein the antigen of SARS-CoV-2 is fused with a membranal protein of the microorganism.

13. The recombinant microorganism of claim 12, wherein the membranal protein of the microorganism is selected from: cotA, cotB, cotC, cotD, cotE, cotF, cotG, cotH, cotK, cotL, cotM, cotS, cotT, cotV, cotW, cotX, cotY, cotZ, cotJA, and cotJC.

14. The recombinant microorganism of claim 13, wherein the membranal protein of the microorganism is cotY.

15. The recombinant microorganism of any one of claims 1 to 14, further displaying a protein adjuvant on the surface of the microorganism.

16. A recombinant microorganism displaying a fusion protein at its surface, wherein the fusion protein comprises: i) a receptor binding portion of a SARS-CoV-2 protein, ii) a multi-epitope chimeric portion of SARS-CoV-2 proteins; and a membranal protein of the microorganism; and wherein the recombinant microorganism is capable of inducing an immune response against a epitopes related to different SARS-CoV-2 proteins infection in a subject.

17. The recombinant microorganism of claim 16, further displaying an enzyme having an enzymatic activity that is absent in the microorganism in the wild.

18. The recombinant microorganism of claim 16 or 17, wherein the microorganism is bacterial.

19. The recombinant microorganism of claim 18, wherein the microorganism is a member of a Bacillus genus.

20. The recombinant microorganism of claim 19, wherein the microorganism is a spore from a member of a Bacillus genus.

21. The recombinant microorganism of claim 20, wherein the Bacillus is selected from Bacillus subtilis, Bacillus circulans, Bacillus clausii, Bacillus amyloliquefaciens and Bacillus velezensis.

22. The recombinant microorganism of claim 20, wherein the Bacillus is Bacillus subtilis.

23. A method for displaying an antigen of SARS-CoV-2 on a surface of a microorganism, the method comprising the steps of:
(i) preparing a vector for microorganism surface display comprising a gene construct, the gene construct comprising: i) a microorganism membrane attachment protein; ii) a gene encoding for an enzymatic activity that is absent in microorganism in the wild, and iii) a gene encoding the antigen of SARS-CoV-2, wherein, when expressed, the gene construct expresses a fusion protein between the a microorganism membrane attachment protein, the gene encoding for an enzymatic activity and the antigen of SARS-CoV-2;
(ii) transforming a microorganism host cell with the vector for microorganism surface display; and
(iii) displaying the antigen of SARS-CoV-2 on a surface of the host cell.

24. The method of claim 23, further comprising, after step ii), selecting transformants without using antibiotics.

25. The method of claim 23 or 24, wherein the gene encoding for an enzymatic activity that is absent in microorganism in the wild is a hydrolase.

26. The method of claim 23 or 24, wherein the gene encoding for an enzymatic activity that is absent in microorganism in the wild is an inulinase.

27. The method of any one of claims 23 to 26, wherein the gene construct further comprises a nucleic acid sequence encoding for at least one rigid linker.

28. The method of any one of claims 23 to 27, wherein the gene construct further comprises nucleic acid sequence encoding for a multi-epitope chimeric portion of SARS-CoV-2 proteins.

29. The method of any one of claims 23 to 28, wherein the microorganism membrane attachment protein is selected from cotA, cotB, cotC, cotD, cotE, cotF, cotG, cotH, cotK, cotL, cotM, cotS, cotT, cotV, cotW, cotX, cotY, cotZ, cotJA, and cotJC.

30. The method of any one of claims 23 to 28, wherein the microorganism membrane attachment protein is cotY.

31. The method of any one of claims 23 to 30, wherein the microorganism is bacterial.

32. The method of any one of claims 23 to 31, wherein the microorganism is a member of a Bacillus genus.

33. The method of any one of claims 23 to 31, wherein the microorganism is a spore from a member of a Bacillus genus.

34. The method of claim 32 or 33, wherein the Bacillus genus is selected from Bacillus subtilis, Bacillus circulans, Bacillus clausii, Bacillus amyloliquefaciens and Bacillus velezensis.

35. The method of claim 34, wherein the Bacillus is Bacillus subtilis.

36. An isolated nucleic acid molecule encoding for a fusion protein, wherein the fusion protein comprises: i) a receptor binding portion of a SARS-CoV-2 protein, ii) a multi-epitope chimeric portion of SARS-CoV-2 proteins; and iii) a membranal protein of the microorganism.

37. A composition comprising the recombinant microorganism according to any one of claims 1 to 22 and a pharmaceutically acceptable diluent, carrier or excipient.

38. A vaccine for prevention or treatment of a Coronavirus infection in a subject, the vaccine comprising an effective amount of the recombinant microorganism according to any one of claims 1 to 22.

39. A vaccine for treatment of a SARS-CoV-2 infection in a subject, the vaccine comprising the composition according to claim 37.

40. The vaccine of claim 38 or 39, further comprising an adjuvant.

41. The vaccine according to any one of claims 38 to 40, wherein the subject is a mammal.

42. The vaccine according to claim 41, wherein the mammal is a human.

43. The vaccine according to claim 41, wherein the mammal is an animal.

44. The vaccine according to any one of claims 38 to 43, wherein the effective amount is an amount sufficient to induce a neutralizing antibody response to the SARS-CoV-2 infection.

45. A method for treating a SARS-CoV-2 infection in a subject, the method comprising administering to the subject the recombinant microorganism according to any one of claims 1 to 22; the composition according to claim 37; or the vaccine according to any one of claims 38 to 44; such that Coronavirus infection is prevented or treated in the subject.

46. A method of inducing immunity against a SARS-CoV-2 infection comprising administering to a subject the recombinant microorganism according to any one of claims 1 to 22; the composition according to claim 37; or the vaccine according to any one of claims 38 to 44; such that the SARS-CoV-2 infection is treated in the subject.
